(19) United States
(12) Patent Application Publication
(10) Pub. No.: US 2025/0010095 A1
(43) Pub. Date: Jan. 9, 2025

(54) MULTIFUNCTIONAL MICROELECTRONICS FIBERS AS IMPLANTABLE BIOELECTRONIC INTERFACES

(71) Applicant: Massachusetts Institute of Technology, Cambridge, MA (US)

(72) Inventors: Polina Olegovna ANIKEEVA, Lexington, MA (US); Atharva Sahasrabudhe, Cambridge, MA (US); Laura Rupprecht, Cary, NC (US); Tural Khudiyev, Cambridge, MA (US); Diego Bohorquez, Cary, NC (US)

(73) Assignees: Massachusetts Institute of Technology, Cambridge, MA (US); Duke University, Durham, NC (US)

(21) Appl. No.: 18/710,934

(22) PCT Filed: Nov. 16, 2022
(86) PCT No.: PCT/US2022/079951

§ 371 Date: May 16, 2024

Related U.S. Application Data
(60) Provisional application No. 63/279,860, filed on Nov. 16, 2021.

(51) Int. Cl. A61N 5/06

(52) U.S. Cl. CPC A61N 5/0622; A61N 2005/0612; A61N 2005/0626; A61N 2005/063; A61N 2005/0632; A61N 2005/0651; A61N 2005/0663

(57) ABSTRACT

Multifunctional microelectronics fiber probes can be chronically implanted in tissue of awake-behaving animals for understanding brain-viscera communication. These fiber probes can be made using thermal drawing to make hundreds of meters of flexible fiber that incorporates features such as light sources, electrodes, thermal sensors, and microfluidic channels in a multilayered configuration. The fiber mechanics can be tuned for two distinct device layouts: (1) higher-modulus, flexible brain fibers for implantation into deep-brain; and (2) soft, compliant gut fibers for implantation into the small intestine. Brain fibers can modulate the deep-brain mesolimbic reward pathway. Gut fibers can perform peripheral optogenetic stimulation of vagal afferents from the intestine to stimulate brain reward neurons. Brain and gut fibers can be connected to a control module, for example, with a coiled, stretchable interconnect that is more flexible and stretches more than even soft gut fibers, in dual-organ (gut-brain) implantation.

### CROSS-REFERENCE TO RELATED APPLICATION(S)

This application claims the priority benefit, under 35 U.S.C. 119 (e), of U.S. Application No. 63/279,860, filed on Nov. 16, 2021, which is incorporated herein by reference in its entirety for all purposes.

### GOVERNMENT SUPPORT

This invention was made with Government support under Grant No. DK127757, R01 NS115025, DK103832, MH122402 awarded by the National Institutes of Health, and under Grant No. EEC1028725 awarded by the National Science Foundation. The Government has certain rights in the invention.

### BACKGROUND

Deciphering how complex, circuit-wide neural signaling in the central nervous system (CNS) and peripheral nervous system (PNS) governs higher-order cognitive functions is a primary goal in neuroscience research. The gut-brain interface exemplifies one such frontier. Recent reports have shown that signaling from the gut affects appetitive decisions and has profound implications on emotions and social interactions. However, the neural bases and mechanisms underlying such brain-viscera communication networks remain elusive. One obstacle to understanding these mechanisms is the lack of versatile technologies that can stably interface with diverse anatomical regions, while being scalable, customizable, and capable of bidirectional recording and stimulation over chronic time scales.

### SUMMARY

Multifunctional microelectronics fiber probes, also called microelectronics-integrated multifunctional fiber probes or multifunctional fiber probes, can stimulate and monitor neural signaling between the brain and the gut or in other tubular organ systems, such as blood vessels or reproductive organs. Multifunctional fiber probes implanted in the brain can also be referred to as brain fiber probes, and multifunctional fiber probes implanted in the gut or gastrointestinal tract (GI) can be referred to as gut fiber probes or GI fiber probes. An example multifunctional fiber probe includes a polymer fiber segment having (i) a proximal end and (ii) a distal end configured to be inserted into tissue. One or more microfluidic channels run along a length of the polymer fiber segment. Each channel has a first opening toward the proximal end of the polymer fiber segment and a second opening toward the distal end of the polymer fiber segment and may have other openings at either end and/or along the length of the polymer fiber segment. Openings at the tip are useful for brain fiber probes, and openings partway along the length are useful for gut or GI fiber probes.

In operation, these microfluidic channels can convey fluid from the first opening to the second opening and vice versa. Flowing fluid from the proximal end to the distal end is useful for injecting nutrients in gut fiber probes. Sucking fluid into the distal end is useful for injecting a virus payload in the brain: the viral payload is sucked up into the microfluidic channel first (also called front loading of the channel) and then pushed out of the the microfluidic channel and into a targeted site in the brain or other tissue. Other implanted fibers cannot suck up biological fluids.

The multifunctional fiber probe also includes electrodes and electrical interconnects running along the length of the polymer fiber segment, solid-state devices disposed along a distal portion of the polymer fiber segment in electrical communication with the electrical interconnects, and a control module at a proximal end of the polymer fiber segment and connected to the electrical interconnects and the electrodes. The electrodes are configured to make electrophysiology measurements of the tissue. The electrical interconnects convey data and/or commands between the solid-state devices, which are configured to interact with the tissue, and the control module, which is configured to record the electrophysiology measurements and to control the solid-state devices. The control module may include a wired or wireless transceiver configured to receive commands from an external device and/or to transmit data to the external device.

The polymer fiber segment may include polycarbonate (PC), styrene-ethylene-butylene-styrene (SEBS), cyclic olefin copolymer (COC), cyclic olefin co-polymer Elastomer (ECOC), polymethylmethacrylate (PMMA), polyphenylsulfone (PPSU), polyetherimide (PEI), and/or conducting polyethylene (CPE), depending in part on the fiber probe's implantation site. Polymer fiber segments used for brain implantation may have a Young's modulus of 2.3-3.0 GPa and a bending stiffness (single cantilever mode) of 25-30 N/m, whereas those used for gut implantation may have a Young's modulus of 5-30 MPa and a bending stiffness (single cantilever mode) of 2-5 N/m.

The solid-state devices can include devices with two or three electrical connections, such as (micro) light-emitting diodes (μLEDs, possibly coated with phosphors), temperature sensors, piezoelectric or pressure sensors, photodiodes, Joule heaters, and/or other devices. The μLEDs can be used for photopharmacology (e.g., with ultraviolet or red light), drug delivery (e.g., from hydrogels or other light-responsive delivery systems), and/or optogenetically modulate tissue (e.g., with pulses of green, blue, and/or red light).

A multifunctional fiber probe can be made by forming a polymer preform defining channels, then thermally drawing the polymer preform to form several hundred meters of multifunctional fiber. The multifunctional fiber is cut or segmented into multifunctional fiber probes. For each multifunctional fiber probe, a distal portion of a conductor running through one of the channels is exposed and electrically coupled to one or more solid-state devices.

Some multifunctional fiber probes may have two or more separate multifunctional fiber segments and implanted at different sites. For example, one segment can be implanted in the gut and another segment can be implanted in the brain. Alternatively, two segments can be implanted in different portions of the brain or in different portions of the gut. These segments can be coupled to the same control module, either directly or through a stretchable interconnect in the form of a helically coiled fiber with embedded metal microwires.

A stretchable interconnect (also referred to as a helical fiber or helical interconnect) can be fabricated using the scalable thermal drawing approach. Such a helical fiber has broken axial symmetry and is produced by engineering an in-situ residual strain on the fiber during the drawing process. This is done by choosing an elastomeric core material such as SEBS within a non-elastomeric cladding material such as PMMA. The intrinsically stored strain energy can produce out-of-plane buckling of the elastomeric core, resulting in a flexible, stretchable helical fiber, for a preform composed of materials (such as SEBS and PMMA) with different Young's moduli and having a non-centrosymmetric cross-sectional geometry.

Highly conducting fiber based stretchable interconnects composed of SEBS cladding with converged tungsten microwires (25 μm diameter) can be produced using scalable thermal drawing. The stiff metal microwires embedded in the helical fiber are now also stretchable due to the geometry of the helical fiber; shaping the geometry of the fiber into a helical form induces stretchability in the composed helical fiber (elastomeric polymer with embedded metal microwires). In one implementation of the helical fiber, the helical fiber or stretchable interconnect is connected on one end to a control module and on the other end to a brain fiber probe (multifunctional fiber probe implanted in the brain). In another example, a 4-5 cm long flexible helical fiber connects a gut fiber probe on the distal end to the control module on the proximal end, making up a total fiber probe length of 8-10 cm. The length of the helical fiber connected to the fiber probe can be selected depending on the location and depth of implantation.

All combinations of the foregoing concepts and additional concepts discussed in greater detail below (provided such concepts are not mutually inconsistent) are contemplated as being part of the inventive subject matter disclosed herein. All combinations of claimed subject matter appearing at the end of this disclosure are contemplated as being part of the inventive subject matter disclosed herein. The terminology explicitly employed herein that also may appear in any disclosure incorporated by reference should be accorded a meaning most consistent with the concepts disclosed herein.

### BRIEF DESCRIPTIONS OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1A shows high-throughput, single-step fabrication of a microelectronics-integrated multifunctional fiber probe made of polymer using thermal drawing. The thermal drawing process yields a several meters-long microscale fiber which can be cut into approximately 1000 rodent-scale fiber probes with tunable mechanics and incorporating solid state microelectronic components.

FIG. 1B shows how a microelectronics-integrated multifunctional fiber probe can can host multiple independently addressable μLEDs for optogenetics, microelectrodes for extracellular electrophysiology, microfluidic channels for gene/nutrient delivery and thermal sensors for tissue thermometry in a miniature footprint.

FIG. 1C shows a plug-and-play wireless control module, device, or interface that enables real-time programmable optical stimulation and data transfer for recording of tissue temperature.

FIG. 1D shows how microelectronics-integrated multifunctional fiber probes together with a wireless module allow for wireless modulation of neural circuits in the deep-brain and/or the small intestine of chronically implanted awake-behaving mice.

FIG. 2A illustrates preform layout and assembly of a microelectronics-integrated multifunctional fiber probe for implantation into the brain (a microelectronics-integrated multifunctional brain fiber probe or brain fiber probe for short) with interconnect channels in the top polycarbonate (PC) layer and precursor to microfluidic channel and recording electrodes in the bottom PC layer.

FIG. 2B is a schematic of the thermal drawing process with simultaneous feeding of metal microwires serving as interconnects (40 μm Ag—Cu) and recording electrodes (25 μm Tungsten).

FIG. 2C is a digital image of a consolidated PC preform with channels for interconnects, electrodes, and microfluidics.

FIG. 2D is an optical micrograph of a cross-section of the drawn microelectronics-integrated multifunctional brain fiber probe, showing highly conserved features.

FIG. 2E shows several meters of as-drawn fiber depicting the scalability of fabrication (left) and an exploded view of acutely bent multifunctional fiber probe surface showing integrated microwires (right).

FIG. 2F shows a fully assembled microelectronics-integrated multifunctional brain fiber probe with input/output (I/O) pins for microelectrodes and μLEDs, access tubing for a microfluidic channel, and a ground wire.

FIG. 2G shows independently addressable blue micro light-emitting diodes (μLEDs) at the distal end of the multifunctional fiber probe.

FIG. 2H shows independently addressable green μLEDs at the distal end of the multifunctional fiber probe.

FIG. 2I shows simultaneous fluid delivery and blue μLED operation highlighting combined optofluidic capability of the multifunctional fiber probe.

FIG. 2J is an electrochemical impedance spectrum of tungsten microelectrode in 1×PBS (n=3) (the inset shows variation of electrode impedance with bending deformation; 45°: P=0.54, F-value=0.05714; 90°: P=0.82, F-value=0.44186 (n=3, One-way ANOVA and Tukey's comparison test).

FIG. 2K is a plot of the optical intensity output (left axis) and efficiency (right axis) of multifunctional fiber probe integrated μLED (λ=470 nm) with varying input electrical power (n=3).

FIG. 2L is a steady-state calibration curve of the multifunctional fiber probe thermal sensor between 32° C. to 42° C. (n=3).

FIG. 2M is a plot of the variation of local temperature recorded using the thermal sensor during operation of an adjacently placed blue μLED in a brain phantom at different frequencies (35.2 mW/mm², 10 ms pulse).

FIG. 2N is a plot of the output speed (left axis) and return rate (right axis) at varying input injection speeds (n=3) for the microfluidic channel in the multifunctional fiber probe.

FIG. 2O is a plot of calculated probe tip displacement at varying displacements of the brain tissue for steel, silica, bare PC fiber probes and PC μLED fiber probe implants.

FIG. 3A depicts a layout of a multi-layered fiber preform for producing a soft microelectronics-integrated multifunctional fiber probe for implantation in the GI or gut (a multifunctional gut/GI fiber probe, gut fiber probe, or GI fiber probe), where styrene-ethylene-butylene-styrene (SEBS) layers for convergence and microfluidics are molded from an inverse metal mold followed by preform assembly and thermal consolidation.

FIG. 3B illustrates a thermal drawing process for transforming the multi-layered fiber preform of FIG. 3A into several meters of soft fiber with simultaneous feeding of interconnect microwires (40 μm Ag—Cu).

FIG. 3C is a digital image showing the cross-section of a fully assembled and consolidated gut fiber preform.

FIG. 3D is a cross-sectional micrograph of the GI fiber probe highlighting conserved features.

FIG. 3E is a digital image of several meters of as-drawn GI fiber wrapped around a spooler, demonstrating scalable fabrication.

FIG. 3F is a digital image of a fully assembled, multifunctional GI fiber probe highlighting the I/O pins and microfluidic access tubing. In this figure, the fully assembled GI or gut fiber probes are ˜8.5 cm long. The inset shows (left) three green and (middle) blue μLEDs on the distal end of the fiber probe and (right) dual optofluidic modality where a bolus of de-ionized (DI) water ejects out during simultaneous operation of a blue μLED.

FIG. 3G is a plot of cyclic buckling of the GI fiber probe over 10⁴ cycles at 1 mm (broader shading) and 5 mm (narrower shading) lateral displacements and corresponding normalized light output from fiber probe μLED (n=3).

FIG. 3H is a plot of output intensity versus radius of curvature from fiber probe μLEDs subjected to deformations at 90° (upper trace) and 180° (lower trace).

FIG. 3I is a plot of bending stiffness versus frequency for fiber probes with SEBS cladding (lower trace, SEBS fiber probe) and PC cladding (upper trace, PC fiber probe) with identical cross-sections in comparison to 400 μm silica fiber probe (dotted) in single cantilever mode.

FIG. 3J shows FEM simulated stress distribution profiles in SEBS (bottom) and PC (middle) fiber probes with identical cross sections in comparison to a silica fiber probe (top).

FIG. 3K shows that ex-vivo intraluminal implantation of rigid silica fiber probe results in rupture and perforation of intestinal tissue (left) while soft GI/gut fiber probes can be implanted without tissue damage (right).

FIG. 3L is a plot of FEM simulated elastic strain versus radius of curvature in Ag—Cu interconnects.

FIG. 3M shows strain distribution profiles in a GI fiber probe bent at different radii of curvature.

FIG. 3N is a plot of microfluidic output speed (left axis) and return rate (right axis) versus injection speeds relevant to intragastric infusions in a straight and bent geometry.

FIG. 4A shows a fully-assembled microelectronics-integrated multifunctional fiber probe for dual GI-brain implantation (dual GI-brain fiber probe for short) comprising a stretchable interconnect in the form of a helically coiled fiber with embedded metal microwires (aka a helical fiber), which can be connected to the fiber segment for implantation in the brain and/or to the fiber segment for implantation in the gut.

FIG. 4B shows the preform for making a stretchable interconnect/helical fiber with PMMA cladding surrounding a SEBS core.

FIG. 4C illustrates thermal drawing of the stretchable interconnect preform, showing pre-stretching of the SEBS core in PMMA cladding, followed by de-cladding or mechanical removal of the PMMA, resulting in a de-cladded, helical SEBS fiber.

FIG. 4D shows the stretchable interconnect (helical fiber) with embedded microwires.

FIG. 4E shows the stretchable interconnect of FIG. 4D being stretched.

FIG. 4F shows a fully-assembled dual GI-brain fiber probe with a stretchable interconnect (helical fiber) as implanted into the brain and gut of a mouse model.

FIG. 4G shows the schematic of a gut fiber implantation in the proximal duodenum of a mouse and the schematic of a stereotaxic brain implantation, showing the connected helical interconnect and also-implanted gut fiber.

FIG. 4H shows stress-strain curves for the helical fiber compared to its straight counterpart, demonstrating that the helical fiber geometry increases the fiber probe's stretchability/flexibility.

FIG. 4I shows I-V characteristics of a μLED connected to a helical fiber for varying percentages of strain applied to the stretchable interconnect.

FIG. 4J shows consistent resistance values after repeated strain cycles on the helical fiber, suggesting that strain cycles do not impact the electrical properties of the metal microwires embedded in the helical fiber.

FIG. 5A is a perspective view of a NeuroStack primary plug-and-play wireless module that allows programmable light delivery and physiological recording.

FIG. 5B is a perspective view of an intensity module for NeuroStack.

FIG. 5C shows independent wireless control of a blue μLED on a brain fiber probe using a NeuroStack module (scale bar=1 cm).

FIG. 5D shows independent wireless control of a green μLED on a brain fiber probe using a NeuroStack module.

FIG. 5E shows independent wireless control of a blue μLED on a GI fiber probe using a NeuroStack module.

FIG. 5F shows independent wireless control of a green μLED on a GI fiber probe using a NeuroStack module.

FIG. 5G is a circuit diagram of NeuroStack highlighting a power management block, temperature sensing block, and intensity control block.

FIG. 5H is a plot showing real-time control of the optical stimulation frequency from 10 Hz to 50 Hz.

FIG. 5I is a plot of real-time control of the optical stimulation duty cycle from 20% to 80%.

FIG. 5J is a plot showing optical pulse shaping through control of pulse rise and fall times from 5 ms to 15 ms.

FIG. 5K shows how the intensity module permits real-time tuning of μLED bias voltage with corresponding snapshots (right) at bias voltages of 0 V (off), 2.4 V, 2.6 V, and 2.8 V.

FIG. 5L illustrates evaluation of wireless temperature sensing performance of NeuroStack under steady-state, showing linear dependence of measured current on surrounding temperature.

FIG. 5M is the wireless temperature sensing performance of the NeuroStack temperature sensor, showing that the dynamic response matches the wired recordings from a commercial thermocouple.

FIG. 6A shows an experimental timeline for in-vivo validation of various fiber probe functionalities.

FIG. 6B shows Cre dependent expression of ChR2-mCherry transcript.

FIG. 6C illustrates one-step gene delivery through microfluidic channel and fiber probe implantation in the same surgical step in the ventral tegmental area (VTA) of TH::Ires-Cre mice.

FIG. 6D is a digital photograph of one-step surgery, with arrows showing the fluid injection setup connected to the microfluidic channel and the implanted fiber probe.

FIG. 6E is digital photograph of a fully recovered animal after surgery carrying a wireless module.

FIGS. 6F-6H show expression of ChR2-mcherry 4-weeks post microfluidic virus injection in the VTA (FIG. 6F shows DAPI, FIG. 6G shows mCherry, and FIG. 6H is a merge of FIGS. 6F and 6G).

FIG. 6I is a plot of electrophysiological recordings of optically evoked neural activity in VTA at 5, 9, 14 and 17 days after virus delivery where optical pulses were delivered through fiber probe μLED (\=470 nm, 10 Hz pulse repetition rate, 5 ms pulse width, 10.2 mW/mm²).

FIG. 6J shows a raw trace of endogenous single unit activity (spikes) recorded from putative VTA neurons at week-4 post implantation.

FIG. 6K shows average spike waveforms from two isolated units (distinct neurons).

FIG. 6L is a plot of wireless intracranial temperature recorded in mice exploring an open field chamber during simultaneous wireless photostimulation with a multifunctional fiber probe (shaded region, 20 Hz, 10 ms pulse width, 0.5 s on duration, 1 s off duration).

FIG. 6M illustrates fiber probe thermal sensors detecting brain hypothermia in the VTA induced by i.p. injection of anesthetic drug mixture in a dose-dependent manner at 30 mg/kg (upper left trace) and 60 mg/kg doses (lower right trace).

FIG. 6N is a schematic depiction of place preference assay for validation of real-time programmable optical stimulation along with a representative digital image of the animal during the behavioral task.

FIG. 6O is a plot illustrating percentage preference in the chamber coupled to rewarding optical stimulation at baseline and on test-day for ChR2-mCherry (left) and mCherry mice (right) for photostimulation at 20 Hz, 10 ms pulse, 0.5 s On duration, 1 s Off duration (ON) versus no photostimulation (OFF); ChR2-mCherry: P=6.52E-4, 1=−5.81, d.f=7; mCherry: P=0.51, 1=−0.69, d.f=7.

FIG. 6P is a plot illustrating percentage preference in the chamber coupled to rewarding optical stimulation at baseline and on test-day for ChR2-mCherry (left) and mCherry mice (right) for phasic photostimulation (40 Hz, 10 ms pulse width, 0.5 s On duration, 1 s Off duration) versus tonic photostimulation (1 Hz, 10 ms pulse width, 1 s On duration, 1 s Off duration); ChR2-mCherry: P=0.00145, 1=−4.74, d.f=8; mCherry: P=0.44634, 1=−0.87, d.f=3.

FIG. 6Q is a plot illustrating percentage preference in the chamber coupled to rewarding optical stimulation at baseline and on test-day for ChR2-mCherry (left) and mCherry mice (right) for blue photostimulation (λ=470 nm, 20 Hz, 10 ms pulse, 0.5 s On duration, 1 s Off duration) versus green photostimulation (2=529 nm, 20 Hz, 10 ms pulse, 0.5 s On duration, 1 s Off duration); ChR2-mCherry: P=2.29E-5, t=−7.96, d.f=9; mCherry: P=0.2023; 1=−1.46, d.f=5.

FIGS. 6R-6T are heat maps tracing the animal position in the experiments of FIGS. 6O-6Q, respectively.

FIG. 7A is a schematic illustration of vagal electrophysiology during optofluidic modulation with a soft, multifunctional GI fiber probe of epithelial neuropod cells in Cck::ChR2 transgenic mice.

FIG. 7B shows that chemical stimulation of Cck cells with microfluidic delivery of sucrose solution (300 mM, 0.2 ml, 3.3 μl/s) increases mean vagal firing rate. Gray bar indicates stimulation epoch.

FIG. 7C shows quantification of peak vagal responses (n=4 mice per group; *P<0.0304 by Kruskal-Wallis test with non-parametric comparisons using Wilcoxon Method).

FIG. 7D shows that optogenetic stimulation of Cck cells with integrated blue μLEDs (λ=470 nm, 40 Hz, 10 ms pulse width) within microelectronics-integrated multifunctional fiber probes increases vagal firing rate while stimulation with green μLEDs (λ=527 nm) has no effect. Gray bar indicates stimulation epoch.

FIG. 7E shows quantification of peak vagal responses (n=3-5 mice per group; *P<0.0367 by Kruskal-Wallis test with non-parametric comparisons using Wilcoxon Method, baseline v/s blue μLED; P=0.0367, baseline v/s green μLED; P=0.1113, blue μLED v/s green μLED; P=0.0200).

FIG. 7F shows a schematic illustration of optogenetic stimulation of vagal afferents in the proximal small intestine of Phox2b::ChR2 transgenic mice enabled by gut fiber probes.

FIG. 7G shows a photograph of an implanted fiber probe in the intestinal lumen of Phox2b::ChR2 mouse with μLEDs in operation.

FIG. 7H shows a photograph of a mouse implanted with a gut fiber probe in the intestinal lumen during postoperative recovery in the homecage.

FIG. 7I shows a pair of chronically implanted mice carrying the NeuroStack module.

FIGS. 7J and 7K show that feeding (J) and drinking (K) behaviors are not affected in mice upon chronic implantation of gut fiber probes in the small intestine.

FIGS. 7L and 7M are plots of percentage preference in the chamber coupled to rewarding optical stimulation at baseline and on test day for Phox2b::ChR2 (L), and control littermate (M) mice at different wireless photostimulation conditions.

FIGS. 7N and 7O are representative heat-maps of mouse position corresponding to the assays summarized in FIGS. 7L and 7M.

FIG. 8A illustrates optogenetic control of duodenal Cck+ cells with implanted gut fiber in the duodenum.

FIG. 8B shows the experimental timeline for evaluating feeding behavior in chronically implanted mice in a homecage feeding assay with ad-libitum access to standard chow.

FIG. 8C shows total chow intake measured over 3 hours for Cck::ChR2 mice that received wireless optical stimulation (20 Hz, 10 ms pulse, 0.5 s ON, 1 s OFF) with a blue wavelength or control green wavelength (within-subject design, n=4 mice, significant effect of time [p=0.0003], significant effect of stimulation [p<0.0001], and significant time*stimulation interaction [p=0.0086], posthoc paired t-test between stimulation at each time point: 1 h-p=0.0161, 2 h-p=0.0376, 3 h-p=0.0044). Inset shows digital photo of an implanted animal connected to wireless module consuming chow diet in homecage.

FIG. 8D shows total chow intake measured over 3 hours for control mice lacking ChR2 expression that received wireless optical stimulation (20 Hz, 10 ms pulse, 30 min) with a blue wavelength or control green wavelength (within-subject design, n=4 mice, significant effect of time [p=0.0020], but no significant effect of stimulation [p=0.4975] or time*stimulation interaction [0.8906]). All lines and markers represent mean values, and all shaded areas and error bars represent s.d. (**P<0.01, ***P<0.001; paired samples t-test).

FIG. 8E illustrates optogenetic control of ileal Pyy+ cells with implanted gut fiber in the ileum.

FIG. 8F shows the experimental timeline for evaluating feeding behavior in chronically implanted mice in a homecage feeding assay with ad-libitum access to Ensure solution (high fat and carbohydrate solution).

FIG. 8G shows Ensure consumption monitored for 60 min at 5 min intervals for Pyy::ChR2 mice during concomitant wireless optical stimulation (20 Hz, 10 ms pulse, 0.5 s ON, 1 s OFF) with either blue wavelength or control green wavelength (within-subject design, n=4 mice, significant effect of time (p<0.0001), stimulation (p<0.0001), and time*stimulation interaction (p<0.0001).

FIG. 8H shows cumulative Ensure intake at the end of a 60 min period for Pyy::ChR2 mice (within-subject design, n=4 mice, posthoc paired t-test, p=0.0381).

FIG. 8I shows Ensure consumption monitored for 60 min at 5 min intervals for control mice that lacked ChR2 expression during concomitant wireless optical stimulation (20 Hz, 10 ms pulse, 0.5 s ON, 1 s OFF) with either blue wavelength or control green wavelength (within-subject design, n=4 mice, significant effect of time [p<0.0001], significant effect of stimulation [p=0.0160], but no significant time*stimulation interaction [p=0.5796]).

FIG. 8J shows cumulative Ensure intake at the end of 60 min period for control mice (within-subject design, n=4 mice, posthoc paired t-test, p=0.4639). All lines and markers represent mean values, and all shaded areas and error bars represent s.d. (**P<0.01, ***P<0.001; paired samples t-test).

### DETAILED DESCRIPTION

Here we introduce microelectronics-integrated multifunctional fiber probes that can interface stably with diverse anatomical regions, including the brain and gastrointestinal (GI) tract. Multifunctional fiber probes implanted in the brain can also be referred to as brain fiber probes, and multifunctional fiber probes implanted in the gut or GI can be referred to as gut fiber probes or GI fiber probes. Other multifunctional fiber probes are dual brain-GI fibers probes with two fiber segments, both coupled to the same control module, with one configured to be implanted in the brain and the other configured to be implanted in the gut or GI tract. These multifunctional fiber probes are scalable, customizable, and capable of bidirectional recording and stimulation over chronic time scales.

Our wireless multifunctional fiber probes act as bioelectronic interfaces that are compatible for chronic implantation with diverse organ systems in freely behaving mice. We leverage a single-step, scalable fiber drawing process to produce hundreds of meters of microscale polymer fiber that can integrate solid-state semiconductor chips along their surfaces in a single production run. Coupled together with deterministic tunability of fiber mechanics, this approach offers unprecedented design flexibility which is demonstrated by producing stiff yet flexible multifunctional fiber probes for the brain (brain fiber probes) and soft compliant fiber probes for the gut (gut/GI fiber probes).

We also overcome outstanding challenges associated with thermal drawing process (TDP). For example, thermally drawn devices have tip-localized functionality, use passive components for sensing/actuation, and are incompatible with tether-free operation. By embedding microelectronics in the fiber probes, we break their axial redundancy and unlock new actuation (lateral optical illumination) and sensing modes (thermal sensors), which can be further extended in scope using commercial off-the-shelf components. We show that fiber probe implants can be controlled wirelessly with modules attached to their proximal ends.

This complete platform addresses several limitations of other technologies, including the conventional cleanroom-based microfabrication techniques that are currently used for producing wireless multifunctional neural interfaces. Microfabrication typically involves a series of lithography, etching and transfer printing steps in resource-intensive cleanroom settings. Rapid customization of microfabricated device layouts becomes prohibitive as each pattern requires design of a new photomask and optimization of several physical/chemical steps. Moreover, lithography based thin-film processes also preclude monolithic integration and instead require manual assembly of individually fabricated layers to form a multifunctional device stack. Low fabrication throughput and limitations on device lengths are other associated hurdles, especially for deployment in anatomically hard-to-reach sites in the peripheral nervous system (PNS).

We leverage our technological advances to study and wirelessly modulate gut-brain neural circuits in awake-behaving animals. The multifunctionality in brain fiber probes enables several recording and stimulation experiments in-vivo such as gene delivery, dynamic and chronic optoelectrophysiology, single neuron recording, sensing of brain hypothermia, and wireless programmable optical control of reward behavior. Soft, multifunctional GI fiber probes uniquely enable light and chemical delivery to targeted sites in the intestinal lumen for modulation of gut enteroendocrine cells and vagal afferents. These fiber probes create a generalized strategy for gut lumen optogenetics that can be extended to other cell types (e.g., neuropod cells), the enteric nervous system, and the spinal nerve branch innervating the gut. This is not possible with current fiber optic approaches that target cell bodies in the brain stem.

These advanced fiber probes facilitate understanding of how gut sensory function governs emotional states and cognitive processes in the brain in health and disease. This allows researchers to take advantage of widely available transgenic mouse lines and perform direct peripheral optogenetics along the GI tract that bypasses complex retrograde viral injections. The devices and concepts developed here can be directly extended to multimodal neuromodulation of other hollow, tubular organ systems such as blood vessels and reproductive tissues in awake-behaving mice. The successful independent validation of brain and gut fiber probes creates exciting opportunities for their co-implantation into these organs in the same animal to help understand bi-directional signal transduction in associated neural circuits. Thus, the technology disclosed here enable wireless interrogation of brain-viscera neuronal communication networks across multiple modalities in health and disease.

### Biocompatible, Microelectronics-Integrating Multifunctional Fiber Probes

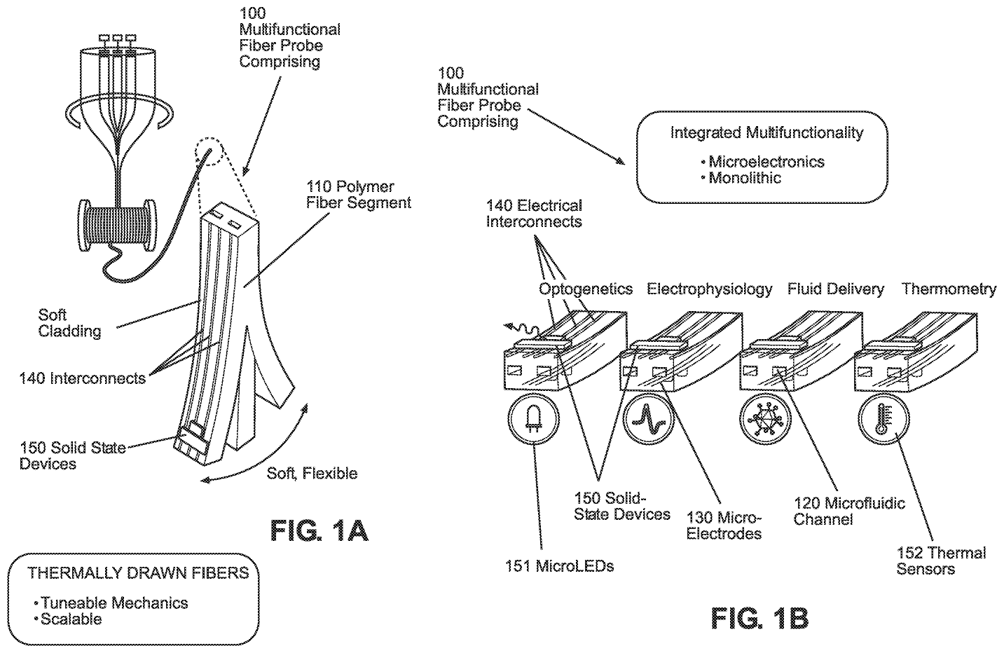

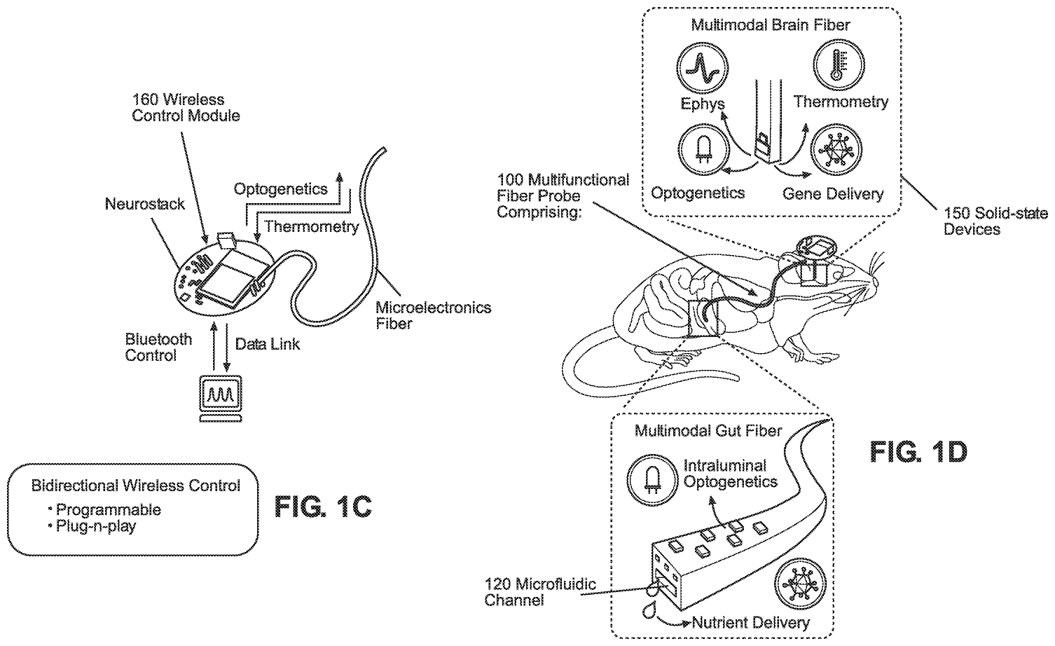

FIGS. 1A and 1B show a wireless microelectronics-integrated multifunctional fiber probe (multifunctional fiber probe) in the form of a polymer fiber probe 100 that incorporates solid-state microelectronic components using the highly scalable thermal drawing process (TDP). We leverage the top-down nature of TDP to produce, in a single step, several hundred meters of microscale fiber 110 (which can be cut into approximately 1000 rodent-scale probes) with interconnects 140 that can host solid state devices 150 which can be: a) surface localized microscale light emitting diodes (μLEDs) 151 for optogenetics; b) microscale thermal sensors 152 for thermometry; c) microelectrodes 130 for single-unit electrophysiology, and d) microfluidic channels 120 for fluid delivery as shown in FIG. 1B. The fiber probe's mechanical properties can be deliberately tuned using thermoplastic elastomers, which allows layouts compatible for implantation in both the deep brain and the anatomically challenging region of the GI tract. The fiber probe can be coupled to and controlled with a plug-and-play wireless control module 160, termed NeuroStack and shown in FIG. 1C, for tether-free animal behavioral studies. NeuroStack 160 permits real-time, programmable light delivery across multiple independent channels and wireless data transfer for recording of local tissue temperature in freely behaving mice.

FIG. 1D shows how these biocompatible multifunctional fiber probes 100 with solid-state devices 150 can be chronically implanted into the brain and the small intestine of awake-behaving mice. The stiff, yet flexible fiber probes designed for the brain (brain fiber probes) can accurately target deep-brain nuclei, such as the ventral tegmental area (VTA). Their microfluidic channels 120 can deliver a viral vector to dopaminergic neurons. They can also record optically evoked and endogenous neural activity, while thermal sensors enable deep-brain thermometry.

Soft, compliant multifunctional gut or GI fiber probes can deliver light and chemicals in the gut lumen, allowing direct modulation of different components of GI neural circuitry, such as the epithelial neuropod cells and upper-gut innervating vagal afferents. Fiber probes implanted in the ventral tegmental area (VTA) and coupled to NeuroStack can optogenetically stimulate dopaminergic neurons to elicit classic reward behavior. Furthermore, the GI fiber probes help reveal that optogenetic stimulation of vagal afferents from the gut lumen also produces a rewarding phenotype, thereby demonstrating for the first-time direct modulation of central nervous system function from the intestine in awake-behaving mice. These applications can be used in systems neuroscience for understanding brain-viscera neural communication networks.

### Results

### Design and Fabrication of Microelectronics-Integrated Multifunctional Fiber Probes for the Brain (Brain Fiber Probes)

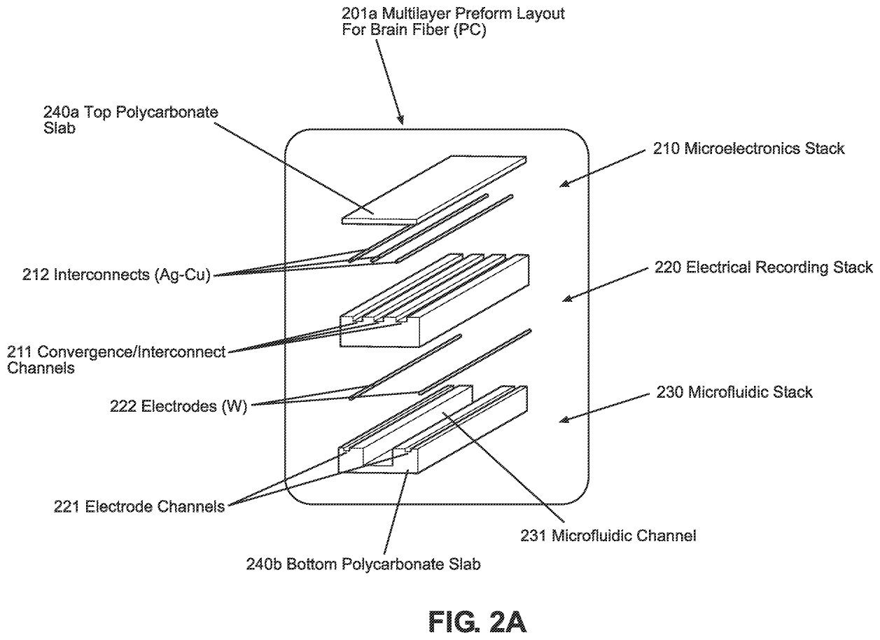

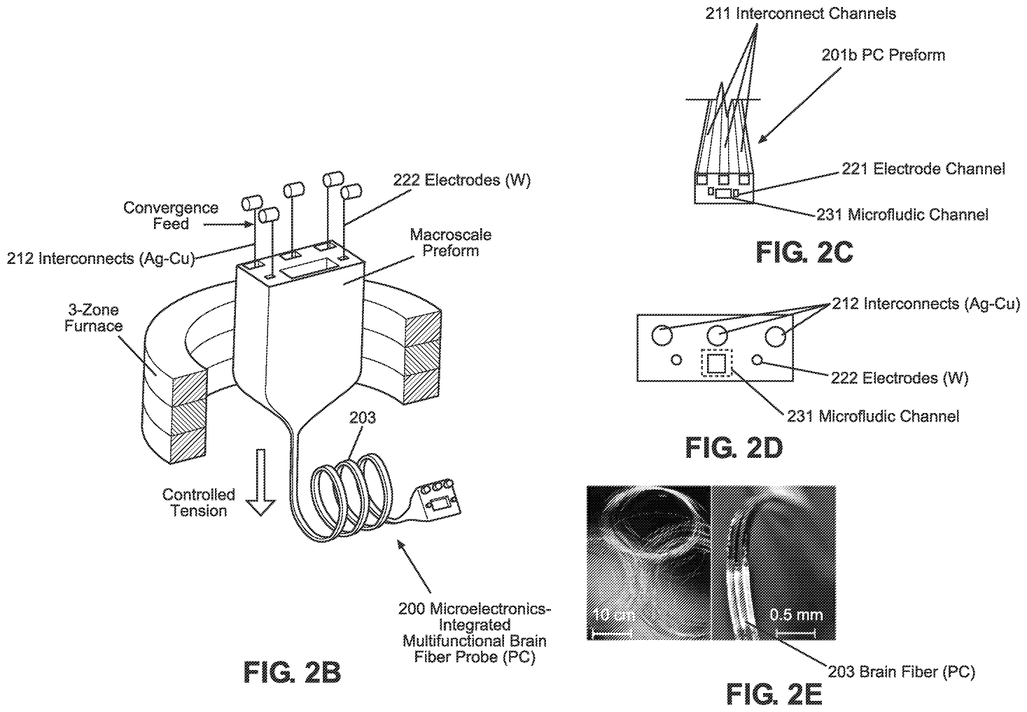

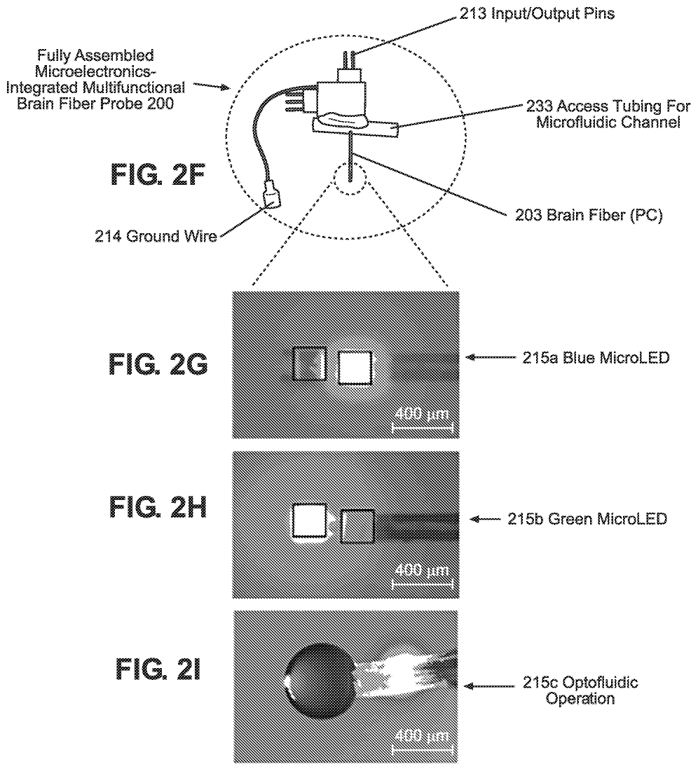

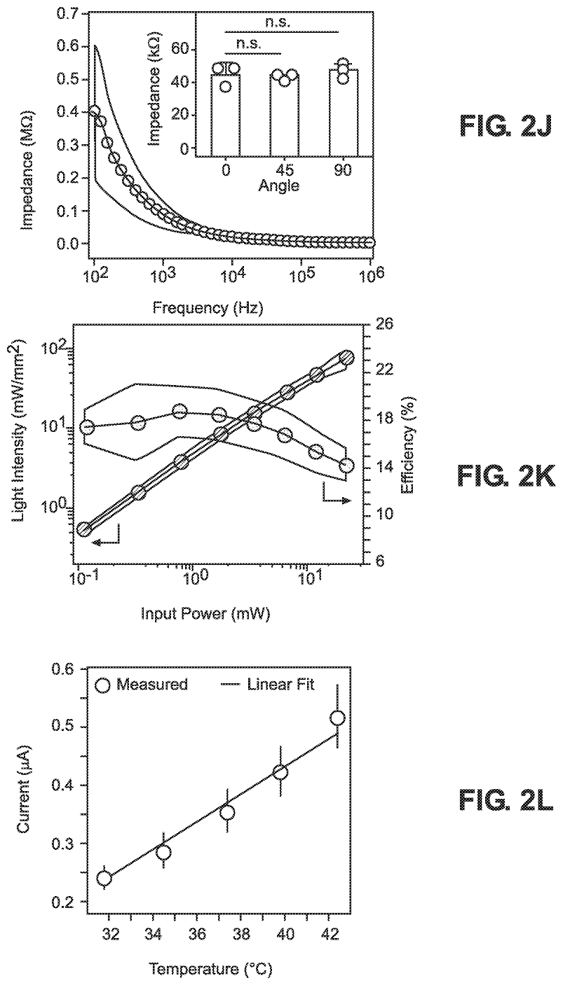

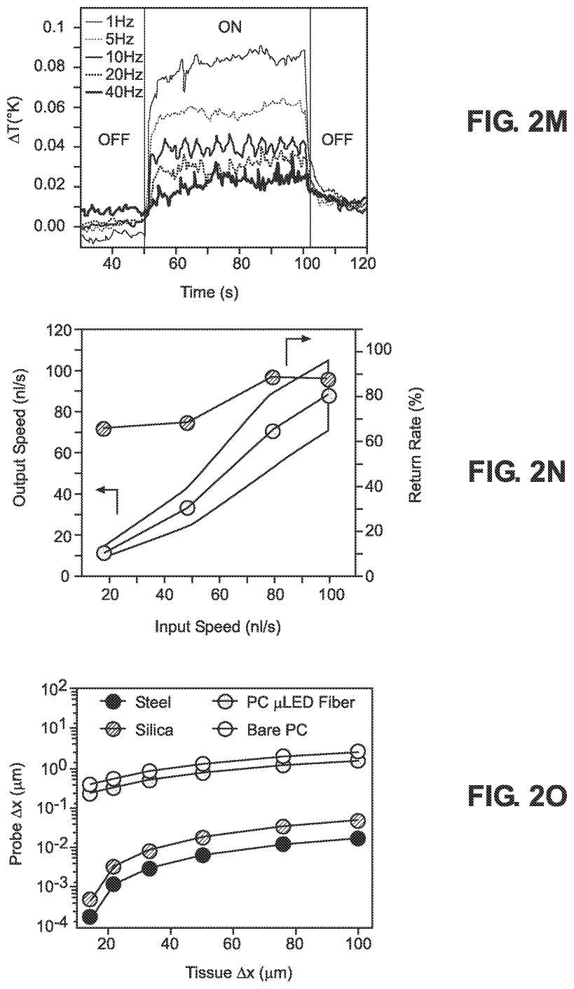

A microelectronics-integrated multifunctional fiber probe for the brain (brain fiber probe) 200 can be made by thermally drawing a multilayer polycarbonate (PC) preform (PC, T_{g}=160° C., E=1.8-3.2 GPa), whose layout 201a is shown in FIG. 2A. The multilayer preform layout 201a can have a microelectronics stack 210, an electrical recording stack 220, and a microfluidic stack 230. The preform, which has a top polycarbonate slab 240a and a bottom polycarbonate slab 240b with microfluidic channel 231, is thermally drawn into a polymer fiber 203 that is several meters long, shown in FIG. 2B, while simultaneously feeding spools of interconnect 212 (Ag—Cu, 40 μm) into convergence/interconnect channels 211 and electrode microwires 222 (Tungsten, 25 μm) into electrode channels 221. The polymer fiber 203 is segmented and coupled to μLEDs and/or other electronic devices to form the brain fiber probe 200.

The overall cross-sectional geometry of the preform 201b, with interconnect channel 211, electrode channel 221, and microfluidic channel 231, shown in FIG. 2C, is conserved during the draw (FIG. 2D) with Ag—Cu interconnects 212 and W electrodes 222, yielding approximately 50 m of polymer fiber 203 (FIG. 2E) with dimensions of 370.7±2.8 μm×190.4±3.4 μm (mean±s.d., n=5 sections). The fiber 203 is part of the fully-assembled microelectronics-integrated multifunctional brain fiber probe 200 (FIG. 2F) with input/output pins 213, ground wire 214, and microfluidic access tubing 233. Blue (λ=470 nm) and green (λ=527 nm) μLEDs 215a and 215b (InGaN, 270 μm×210 μm×50 μm) can be mounted along the fiber probe surface followed by deposition of a roughly 10 μm thick layer of parylene-C as a biofluid barrier coating. μLEDs and/or other semiconductor devices can also be embedded inside a multifunctional fiber probe during the draw itself. In the fiber probe of FIG. 2B, for example, the μLEDs are added after fiber drawing. The μLED chips are reflow-soldered to and held firmly in place by the metal interconnects 211 in the fiber probe. FIGS. 2G-2I show optical micrographs of independently operated fiber probe μLEDs 215a, 215b, and 215c together with microfluidic infusion capability via access tubing 233 in the final fiber probe device. The fiber probe integrated microelectronics can be coupled to a wired or wireless control module 160 (FIG. 1C) through male header pins 213. This module may have a Bluetooth radio for communication as well as a microcontroller and associated circuitry that allows control of fiber probe chips. The module can also host a miniature rechargeable battery for power.

### Electronic, Optical, Fluidic, Mechanical, and Thermal Characterization of Brain Fiber Probes

Incorporation of tungsten microwires in fiber probes afforded low impedance microelectrodes (|Z| of 46.3±6 kΩ at 1 kHz) for in-vivo electrophysiology while still retaining fiber flexibility (FIG. 2J and inset). Long-term stability tests in PBS resulted in a modest increase in |Z| over 7 weeks, and no leakage current was observed through the polymer cladding. The light intensity from integrated blue μLEDs was tunable over a wide range (FIG. 2K) between 2.6 mW/mm² to 68.1 mW/mm², which is sufficient for optogenetic modulation of animal behavior. The robust bonding of μLEDs was confirmed from the stable light output at large bending deformations, while long-term immersion tests in PBS demonstrated functional stability for at least 7 weeks.

We used finite element modelling (FEM) to investigate how optical intensity, illumination depth, and volume varied with distance from the μLED at varying input intensities. From this analysis it is apparent that even a moderate illumination intensity of 30 mW/mm² covers a tissue volume of ˜0.75 mm³ around the fiber probe tip, sufficient for optogenetics in most brain nuclei.

We leveraged the temperature-dependent I-V characteristics of the μLED (InGaN μLED, λ=470 nm) to operate it as a microscale thermal sensor for recording heat dissipation in the tissue during μLED operation. A linear dependence of diode current on temperature defined the sensor calibration curve (FIG. 2L). The sensor detected a maximum temperature rise of 0.085° C. from an adjacent μLED (λ=470 nm, ˜30 mW/mm²) operating at 40 Hz, which is well below the ˜2° C. heating that occurs in human deep brain stimulation paradigm (FIG. 2M). This was corroborated by 3D numerical models that estimated a small temperature rise (0.15° C.) for physiologically relevant optical pulse parameters (30 mW/mm², 10 ms, 20% duty cycle) while the temporal dynamics of the temperature profile confirmed that there was no buildup of dissipated heat in the interpulse region.

FIG. 2N shows that the return rate of fluid infusion through the fiber probe microfluidic channel is consistently high (80-100%) at infusion speeds between 20-100 nl/s. Finally, mechanical properties of the fiber probes were evaluated through bending stiffness measurements in a single cantilever mode. The fiber probes exhibited significantly lower bending stiffness (25-33 N/m) as compared to silica (120 N/m) and stainless steel (150 N/m) implants of similar sizes. The mechanical FEM simulations in FIG. 2O show that the estimated displacement of the fiber probe tip was 2-4 orders of magnitude higher compared to steel and silica, which suggests reduced relative micromotion and potentially greater long-term biocompatibility in the brain.

### Design and Fabrication of Soft Microelectronics-Integrated Multifunctional Fiber Probes for the Gut (Gut Fiber Probes)

Unlike the brain, the gut generally prevents chronic implantation of rigid devices owing to a tortuous, tubular anatomy involving delicate tissue that encases a lumen through which ingested food and fluids pass. Hence, multifunctional fiber probes for the gut (gut fiber probes) are more compliant (e.g., 10-15 times less stiff) than the fiber probes for the brain (brain fiber probes). They also have μLEDs and microfluidic channels that enable site-specific delivery of light and nutrients, respectively, in the intestinal lumen of awake-behaving mice.

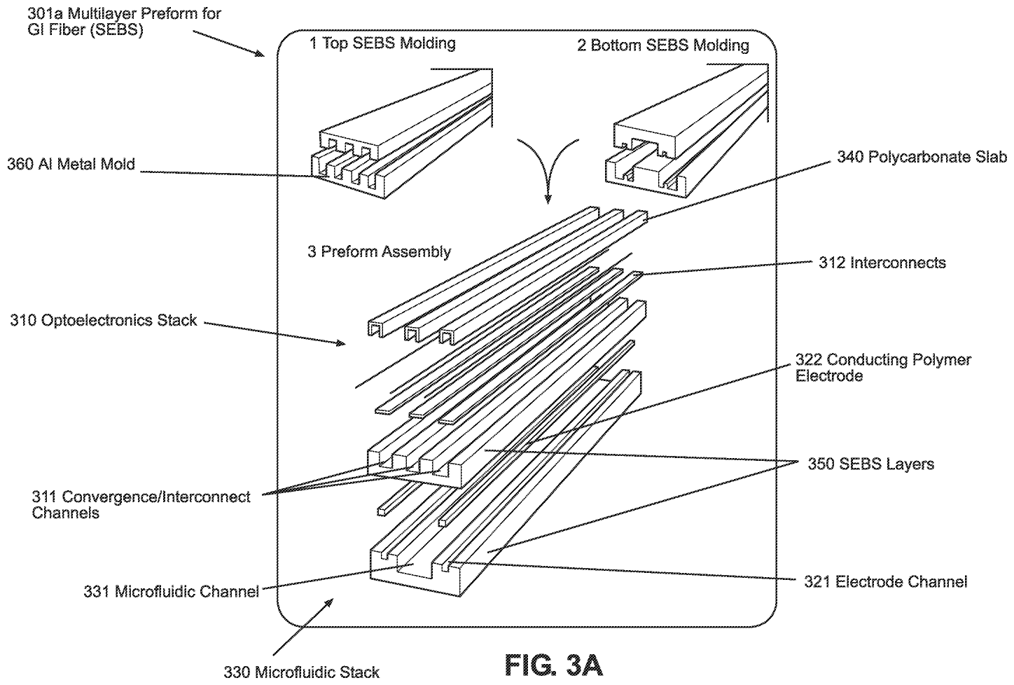

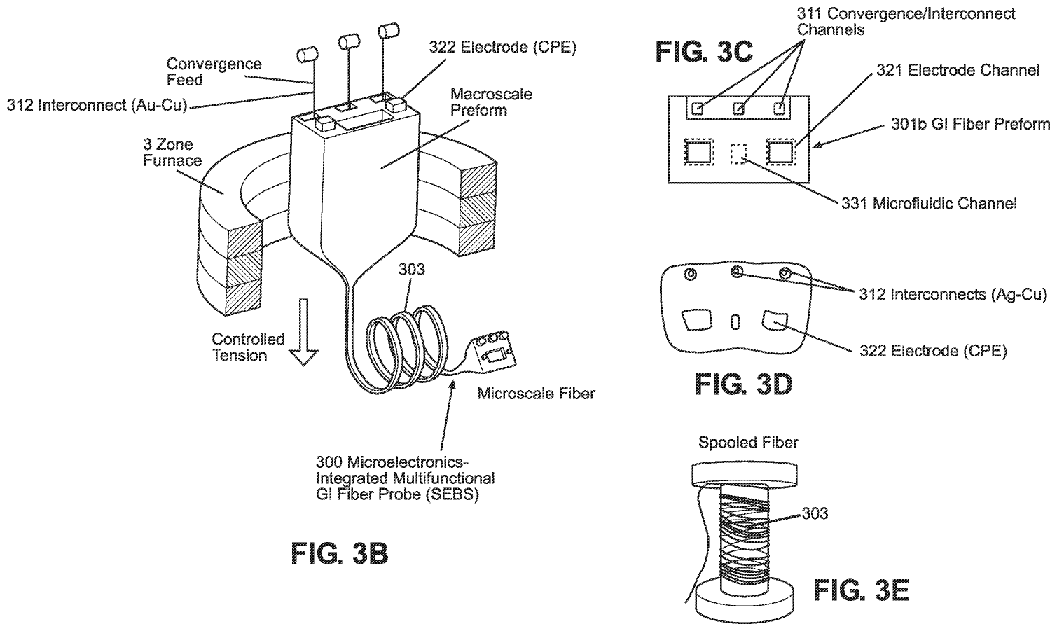

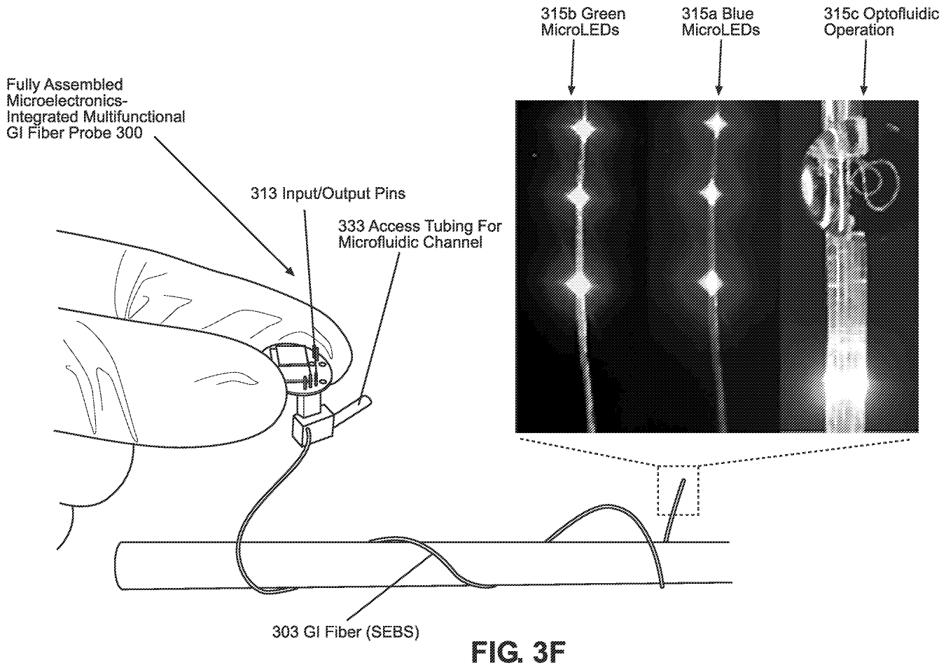

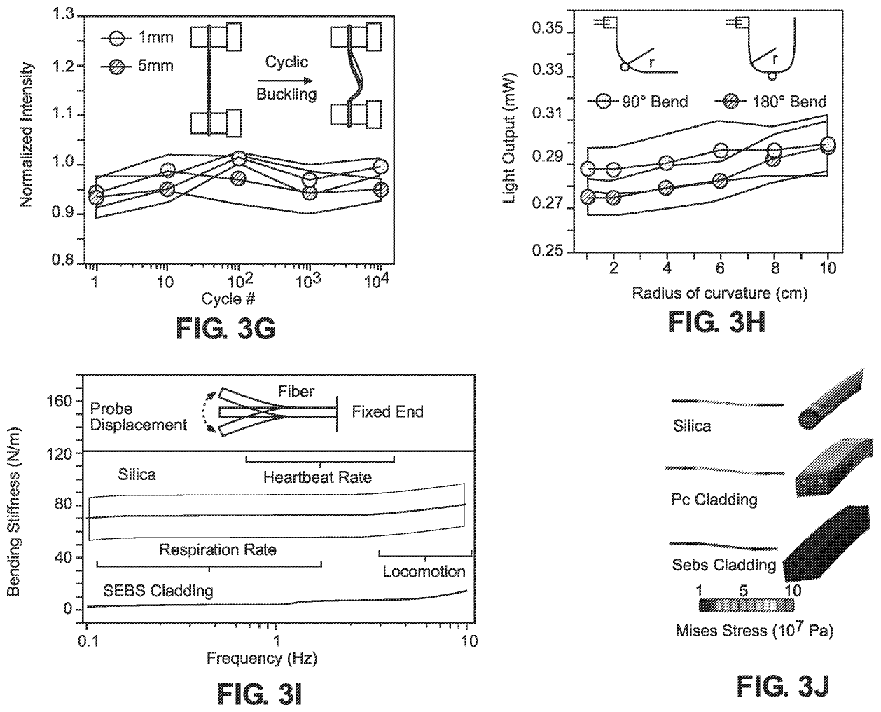

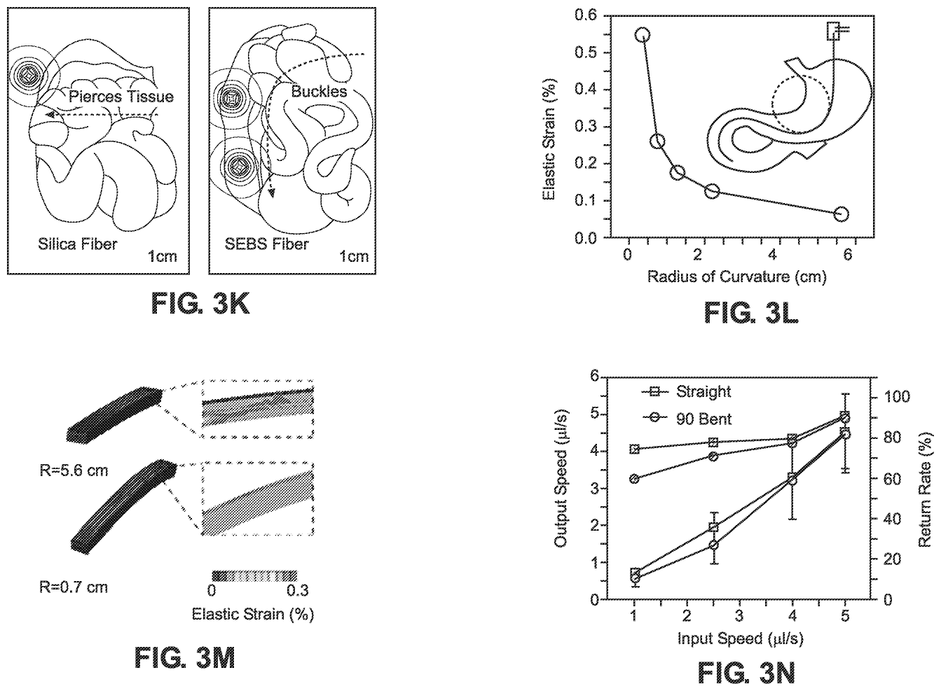

FIG. 3A shows a multilayered preform layout 301a for a multifunctional GI fiber probe. This preform includes thermoplastic poly[styrene-(ethylene-co-butylene-styrene] triblock copolymer (SEBS, T_{g}=140° C., E=3-5 MPa) layers 350. SEBS is approximately 103 times softer than polycarbonate (PC). Top and bottom SEBS preform layers were molded against a metal Al mold 360, then assembled into the preform stack 301a with a polycarbonate slab 340, an optoelectronics stack 310 (with interconnects 312 in convergence/interconnect channels 311 and conducting polymer electrodes 322 in electrode channels 321), and microfluidic stack 330 with microfluidic channel 331. Conducting polyethylene (CPE) 322 was embedded in the fiber probe to provide recording electrodes for making electrophysiological measurements of the gut.

FIG. 3B shows how the preform was drawn into a GI fiber 303 (535 μm×315 μm) while simultaneously feeding interconnect microwires 312 and conducting polymer electrodes 322 to form the microelectronics-integrated multifunctional GI fiber probe 300. FIGS. 3C-3E show that the cross-sectional profile of the GI fiber preform 301b was conserved in the several meters of spooled length of the resulting fiber probe, whose cross-section shows interconnect channels 311, electrode channels 321, and microfluidic channel 331. FIG. 3D shows the cross-sectional profile of the fiber probe with Ag—Cu interconnects 312 and CPE/conducting polymer electrodes 322, whose locations match their corresponding channels as shown in FIG. 3C. FIG. 3F shows the fully assembled microelectronics-integrated multifunctional GI fiber probe device 300 comprising multifunctional GI fiber probes that were approximately 8.5 cm long. In the GI fiber probe shown in FIG. 3F, there are six μLEDs 315a and 315b (three each) located at the distal 2.5 cm ends of the fiber probes. In other examples, the μLEDs can be located anywhere along the fiber length based on the application or organ anatomy. The fiber probe device 300 also has input/output pins 313. Optofluidic operation 315c is depicted in the inset in FIG. 3F. These μLEDs could be operated as two independently addressable sets of three blue 315a and three green 315b μLEDs as shown in the inset of FIG. 3F and a microfluidic outlet 333 0.5 mm posterior to the first μLED pair.

### Optical, Mechanical, Thermal, and Fluidic Characterization of Soft GI Fiber Probes

For optical characterization of GI fiber probes, we first measured the cumulative light output from the three axially distributed μLEDs and their functional stability in PBS over several weeks. The surface-mounted μLEDs also enable a laterally directed illumination profile for the GI fiber probes. This illumination profile allows spatial targeting of gut epithelial cells and vagal afferents from within the lumen, in contrast to a mismatched dorsal-ventral oriented light cone of silica waveguide. We measured the total optical output on the outer surface of the intestinal wall and observed only modest attenuation in optical power in the presence of the tissue. Using FEM simulations, the optical penetration depth in the gut wall was found to be between 1.65 to 2.5 mm, and the total illumination volume was estimated to be between 0.9-8.8 mm³. This is sufficient to broadly cover the subepithelial mucosa layer that receives dense vagal innervation and is situated within 50-100 μm from mucosal membrane. For a range of illumination intensities, the temperature change in the gut wall was negligible, while the μLED separation was sufficient at about 1 mm to prevent co-operative heat buildup. These combined modelling and experimental studies provide quantitative insights into the illumination and thermal characteristics of GI fiber probes for gut optogenetics.

FIGS. 3G and 3H show cyclic buckling and bending test results. These results confirmed the functional integrity of the multifunctional GI fiber probe at extreme deformations, which is especially useful because the gut is a highly mobile anatomical region. The overall bending stiffness of the GI fiber was found to be as low as 2-5 N/m across a range of frequencies, much lower compared to other fibers of identical cross-section. The stiffness of PC fibers was between 70-75 N/m and the stiffness of 400 μm silica waveguide fibers was 120 N/m; these were 15-20 times stiffer than the GI fiber made of SEBS, as shown in FIG. 3I. The rigid silica fiber punctured the mucosal membrane during ex-vivo intraluminal implantation and thus is unsuitable for in-vivo use, whereas the soft GI fiber readily negotiated the lumen curvature without damaging the epithelial tissue, as shown in FIGS. 3J and 3K.

Since surgical implantation of the GI fiber probe involves bending the fiber portion of the fiber probe at acute radii, we simulated strain distribution in the copper interconnects. The copper interconnects have the lowest yield strain among the fiber probe constituents, which was below the elastic limit of 0.3% for radii>0.5 cm, as shown in FIGS. 3L and 3M. FIG. 3N shows the microfluidic capability of GI fiber probes at various injection speeds relevant to intestinal nutrient delivery. It shows consistently high return rates and efficiency under straight and bent geometries.

### Design and Fabrication of Stretchable Helical Interconnects for GI-Brain Implantation

Simultaneous multi-organ (for example, gut-brain) implantation of multifunctional fiber probes can motivate future functional studies of interoceptive neural circuits with these or similar multi-site wireless devices. Such a device can include both gut and brain implantable fiber probes connected to a common wireless module via the same I/O interface. Unfortunately, attaching both the brain and gut fiber probes directly to the same I/O interface can restrict the fiber probes' free motion in three-dimensional space. This, in turn, can prevent accurate positioning of the fiber probe over a brain region of interest, which is helpful for precise targeting in rodent brain surgeries.

To address these challenges, we use a stretchable interconnect in the form of a helically coiled fiber (also called a stretchable interconnect, helical fiber, or helical interconnect) with embedded metal microwires using the scalable thermal drawing approach. Such a non-conventional helical fiber with broken axial symmetry can be produced by engineering an in-situ strain during the drawing process by appropriate choice of elastomeric core and non-elastomeric cladding materials. Accumulation of residual strain in the elastomeric core when it is drawn within a non-elastomeric cladding material, potentially originating from differences in melt viscosities at the drawing temperature, can produce out-of-plane buckling of the elastomeric core if it is composed of materials with differing Young's moduli and has a non-centrosymmetric cross-sectional geometry.

With this fabrication process, we produced highly conducting stretchable interconnects/helical fibers composed of SEBS cladding with converged/embedded tungsten microwires (25 μm diameter). Stretchability is induced in these helical fibers due to the manipulation of their geometry into the helically coiled shape. Even if included in a soft GI fiber probe made of soft SEBS polymer, the embedded metal microwires would still have limited stretchability. Conversely, the same stiff metal microwires, when embedded in a pre-strained SEBS fiber that is released from a PMMA outer cladding, assume the same helical shape as the SEBS fiber. When the helical SEBS fiber is then stretched, its embedded metal microwires also stretch, resulting in more freedom of motion and allowing both brain and GI fiber probes to be implanted and connected to the same I/O interface.

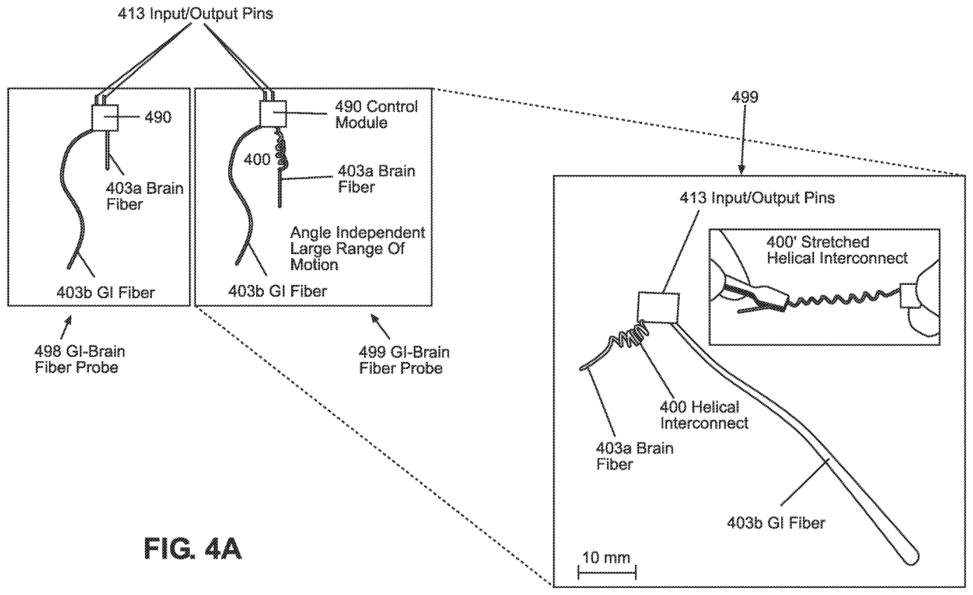

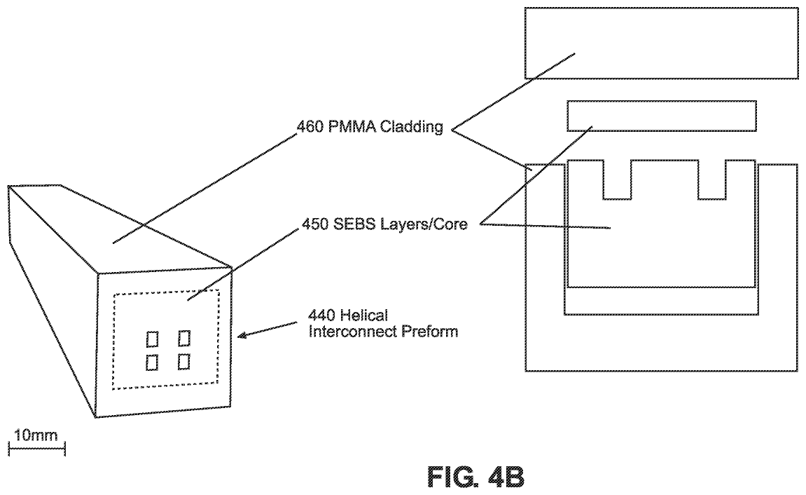

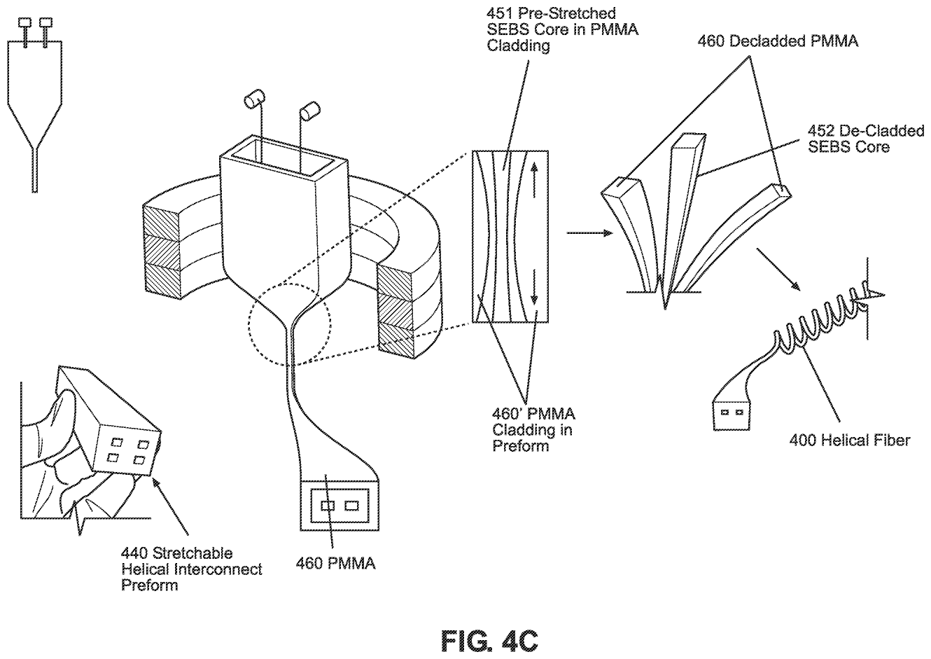

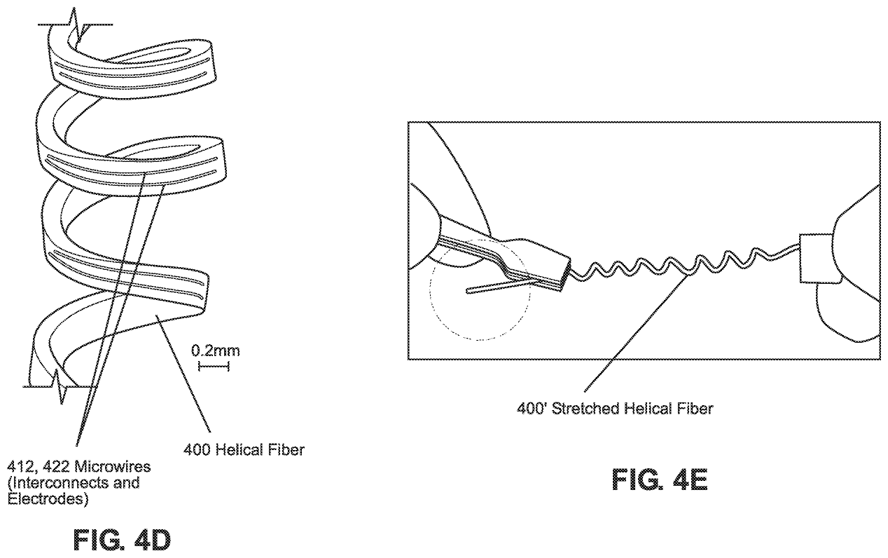

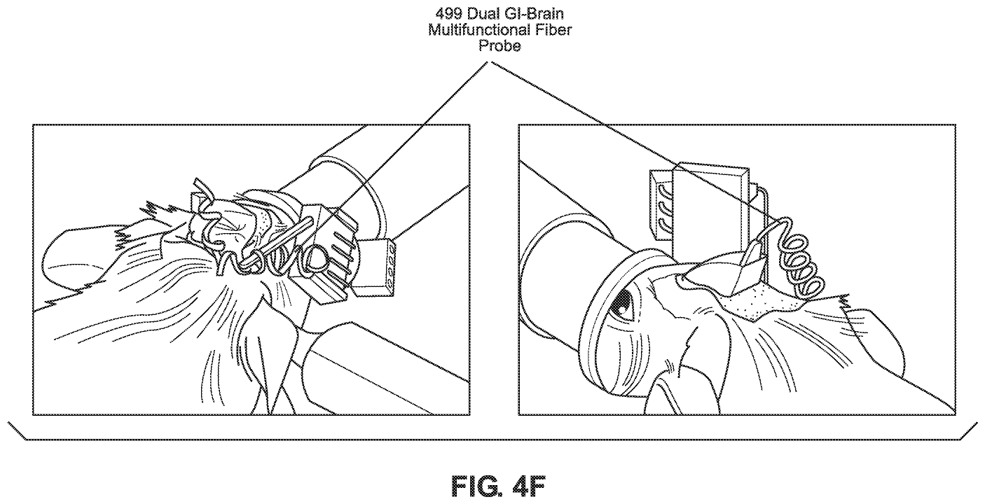

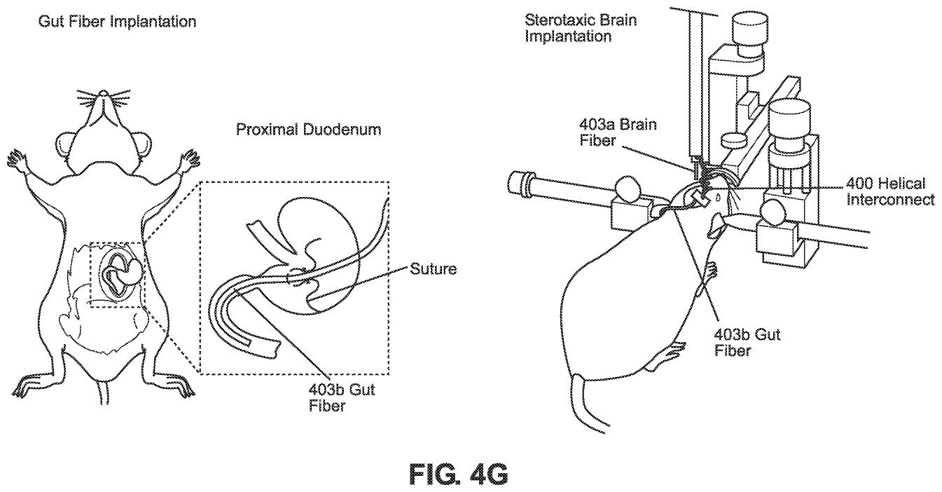

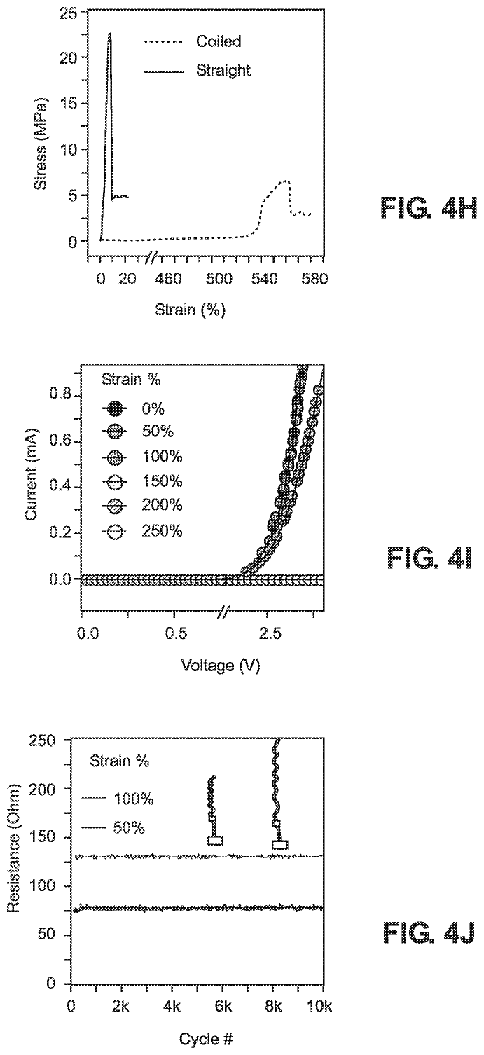

FIG. 4A shows a microelectronics-integrated multifunctional GI-brain, dual-function fiber probe 499 with a stretchable interconnect or helical fiber 400 that includes embedded metal microwires. The helical fiber 400 can be connected to brain fiber 403a and/or gut fiber 403b. Input/output pins 413 connect the fibers to a control module 490 (e.g., a NeuroStack module as described in greater detail below). The helical fiber connects to the brain fiber 403a on one end and to the I/O interface on the other end, and the gut fiber 403b is also connected to the same I/O interface. The left side of FIG. 4A also shows a fully-assembled microelectronics-integrated multifunctional GI-brain, dual-function fiber probe 498 without a stretchable interconnect.

In another implementation, 4-5 cm of the 8-10 cm length of implanted gut fiber probe can be helical fiber to allow for more flexibility in motion. Helical fiber can form part of the brain fiber probe, the GI fiber probe, or both, and the total length of the helical fiber to be used can be adjusted depending on the location and depth of implantation. The form 400′ of helical fiber 400, in the inset of the right side of FIG. 4A, shows the helical fiber while it is being stretched.

FIG. 4B shows the helical fiber/stretchable interconnect preform 440 for producing a stretchable interconnect or helical fiber 400. The preform is made of non-elastomeric polymethylmethacrylate (PMMA) cladding 460 surrounding an elastomeric SEBS core 450, with channels through which metal microwires are also drawn. Interconnect preform 440 incorporates differing stresses as a result of the pre-stretching of the elastomeric SEBS core (see pre-stretched SEBS core 451 in FIG. 4C), utilizing its different Young's modulus compared to the Young's modulus of the outer PMMA cladding to produce a SEBS fiber that coils into a helical shape once the drawing is completed and the outer PMMA is removed or de-cladded.

FIG. 4C illustrates thermal drawing of the stretchable interconnect preform 440, with a cross-sectional inset showing a pre-strained SEBS core 451 within an outer PMMA cladding 460′. Following the thermal drawing process, the PMMA cladding 460′ is mechanically peeled off, releasing the de-cladded inner SEBS core (SEBS fiber) 452. The removal of the outer PMMA cladding results in an out-of-plane buckling of the inner, de-cladded SEBS core 452 along with its embedded metal microwires, thereby producing the helical fiber or stretchable interconnect 400. The helical fiber enables freedom of motion in the implantation of both brain and gut fiber probes.

FIG. 4D shows a close-up image of the the stretchable interconnect/helical fiber 400 with embedded metal microwires 412 (interconnects) and 422 (electrodes). The stretched helical fiber 400′, shown in FIG. 4E, shows that the use of mechanical stress/strain in the thermal drawing process for the fiber results in a modified geometry (helically coiled shape), which in turn increases the flexibility or stretchability of the fiber along with its embedded metal microwires. This increased stretchability allows the helical fiber to be used in simultaneous multi-organ implantation of multifunctional fiber probes, including in gut-brain implantation.

FIG. 4F shows a fully-assembled dual GI-brain microelectronics-integrated multifunctional fiber probe (with helical fiber/stretchable interconnect) 499 as implanted into the gut and brain of a mouse model. The brain fiber, implanted into the brain of the mouse utilizing a stereotactic frame, is connected to the helical fiber, which is in turn connected to the I/O interface and the control module. FIG. 4G shows schematics of the implantation of gut fiber 403b in the proximal duodenum of a mouse and of the stereotaxic brain implantation of brain fiber 403a connected to helical interconnect 400 using a sterotaxic frame; the gut fiber 403b is also shown in the overall dual GI-brain implantation set-up.

FIG. 4H shows a plot of the stress-strain curves comparing the strain behavior of the helical fiber/stretchable interconnect with its straight counterpart, demonstrating the impact of fiber geometry on its flexibility. The helical fiber's geometry allows it to be much more flexible and stretchable than its straight counterpart, even if they are both made from the same soft material (SEBS). Similarly, the stiff metal microwires embedded in the SEBS fiber behave as a more flexible material due to the helically coiled geometry of the helical fiber/stretchable interconnect.

FIG. 4I shows measured I-V characteristics for different strain percentages of a μLED (InGaN) connected to a helical fiber. This defines the functional threshold at which the interconnect transitions from an elastic to a plastic deformation, causing an open circuit.

FIG. 4J shows that repeated cycles of stretching do not change the resistivity of the metal microwires in the helical fiber/stretchable interconnect.

NeuroStack enables programmable and bidirectional wireless control of microelectronics-integrated multifunctional fiber probes.

Incorporation of microelectronics in polymer fiber probes provides a unique opportunity for bi-directional wireless operation in the context of animal behavior experiments. To realize this, we designed and assembled NeuroStack 500, which enables programmable wireless optical stimulation across two independent channels and data transfer for temperature recording.

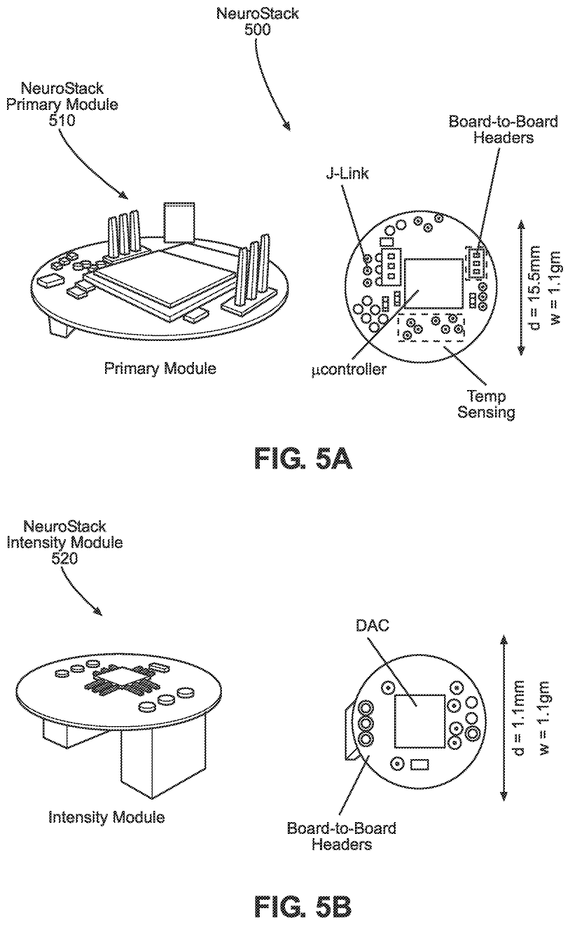

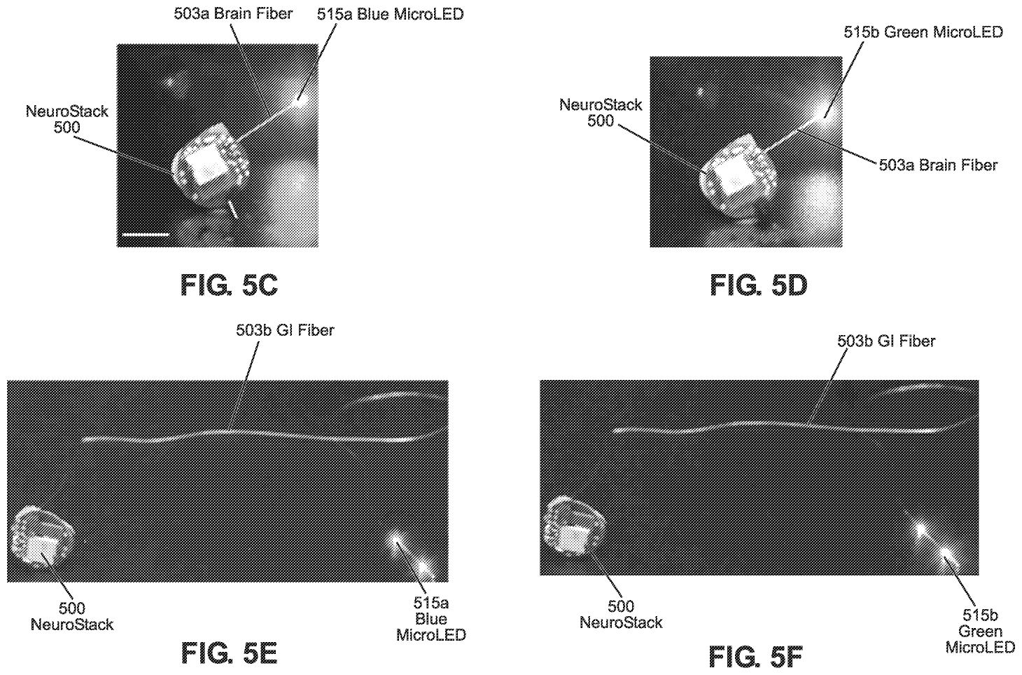

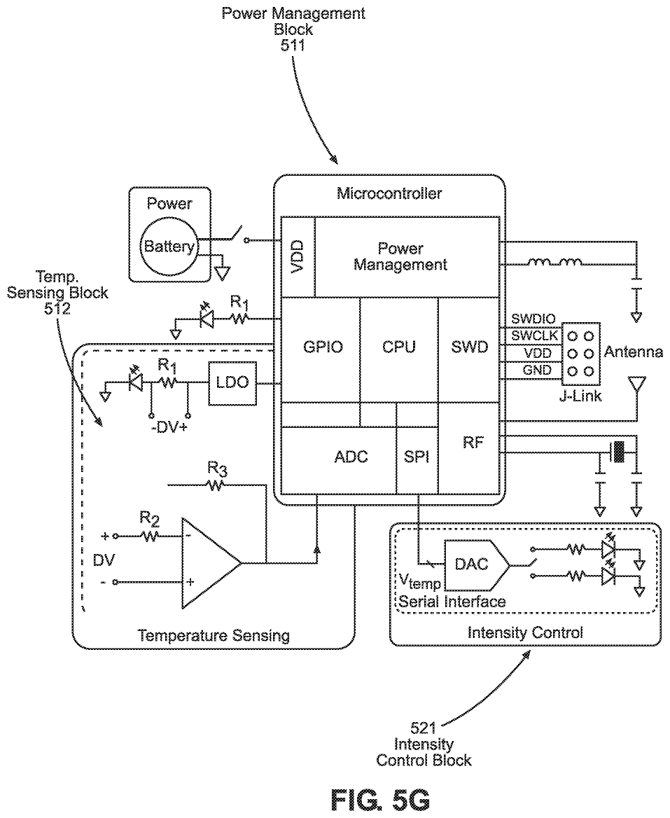

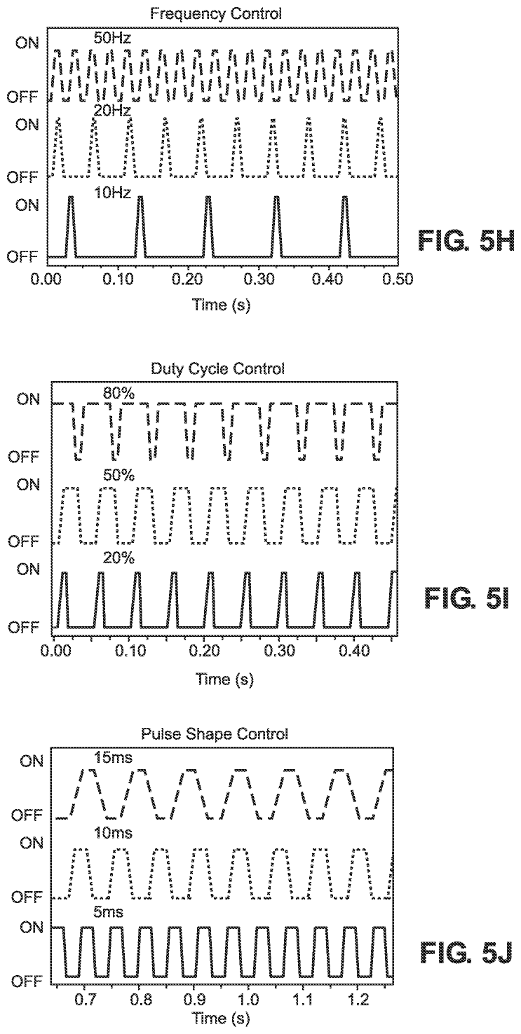

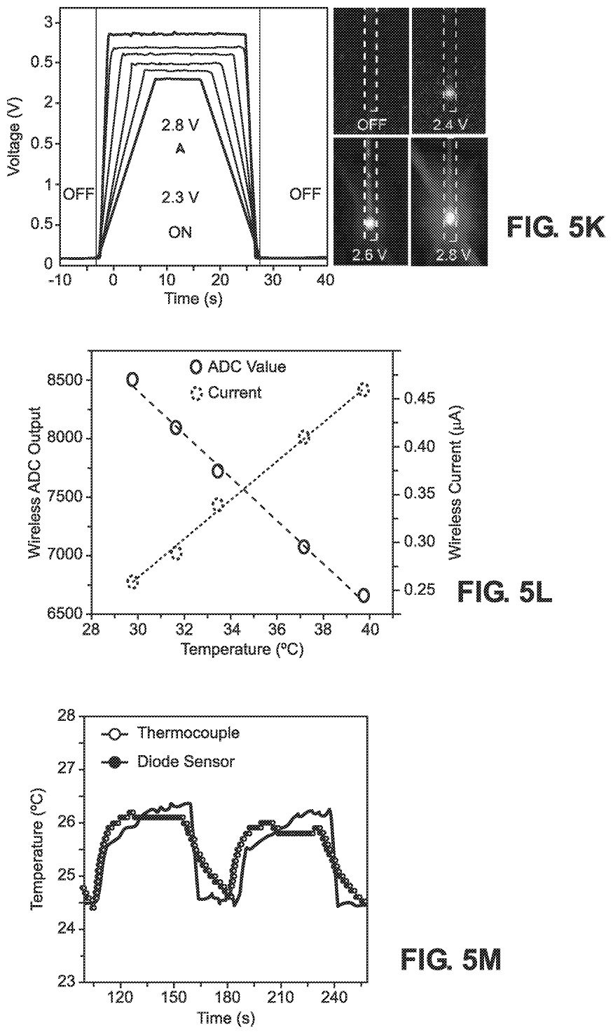

FIG. 5A shows a schematic (left) and a digital photograph (right) of the primary module for NeuroStack 500. The primary module 510 is fully functional as a standalone and performs energy-efficient optical stimulation and wireless data transfer using Bluetooth low energy (BLE) communication protocol. The header pins at the top of the primary module 510 support optional attachment of an intensity stack/module 520, shown in FIG. 5B, which can control intensity levels for photo-stimulation. The intensity stack 520 also allows transient shaping of stimulation pulses, a feature that reduces coupled electromagnetic artifacts in optoelectrophysiology recordings. FIGS. 5C-5F show wireless operation of the independently addressable μLEDs 515a (blue) and 515b (green) on brain (503a) and GI (503b) fibers connected to NeuroStack 500. FIG. 5G shows an electrical layout of the primary module power management block 511, together with temperature sensing block 512 and intensity control circuit block 521.

We characterized the system capabilities of NeuroStack in terms of controlling the frequency, duty cycle, pulse shape, and the intensity of the optical stimulation in real-time from a user-friendly graphical user interface (GUI). The creation of the stimulation pulse includes two phases controlled by software timers. One of the timers manages the change in state between pulsing and resting period, thereby controlling frequency of optical stimulation (FIG. 5H), while the other timer sets the duty cycle during the pulsing period (FIG. 5I). The same principle is used for the intensity control to set the maximum intensity and rise/fall times of the pulses. The step sizes are calculated based on the rise/fall times specified by the user, which can be programmed to range between 1-10 ms. (FIGS. 5J and 5K). Calibration of the fiber probe thermal sensor using NeuroStack was done under steady state between 29° C. to 40° C. The resulting plot of the wireless temperature sensing performance of NeuroStack showed that the measured current has a linear dependence on the surrounding temperature (FIG. 5L) while the dynamic response matched closely to that of wired recordings from a commercial thermocouple (FIG. 5M).

### Multimodal Interrogation of Deep-Brain Dopaminergic Neurons in Awake-Behaving Mice

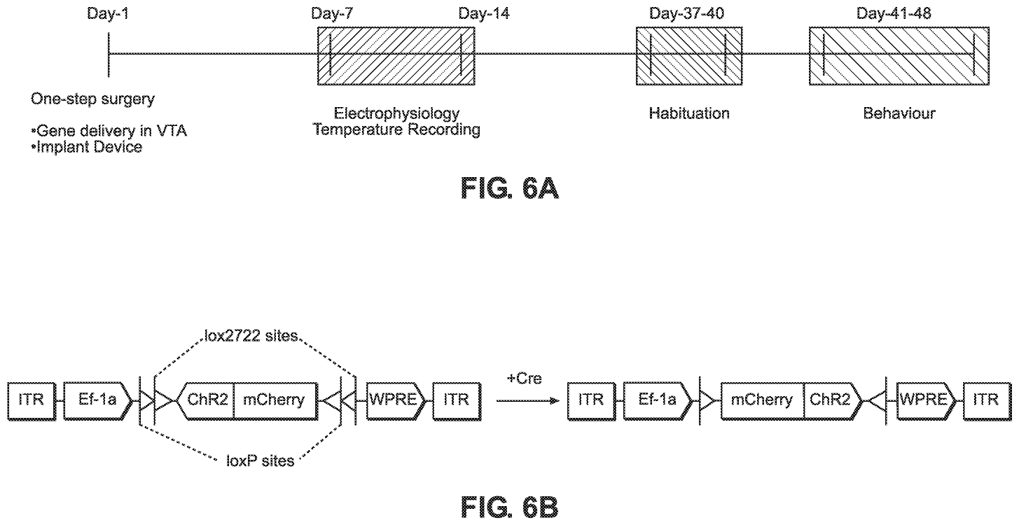

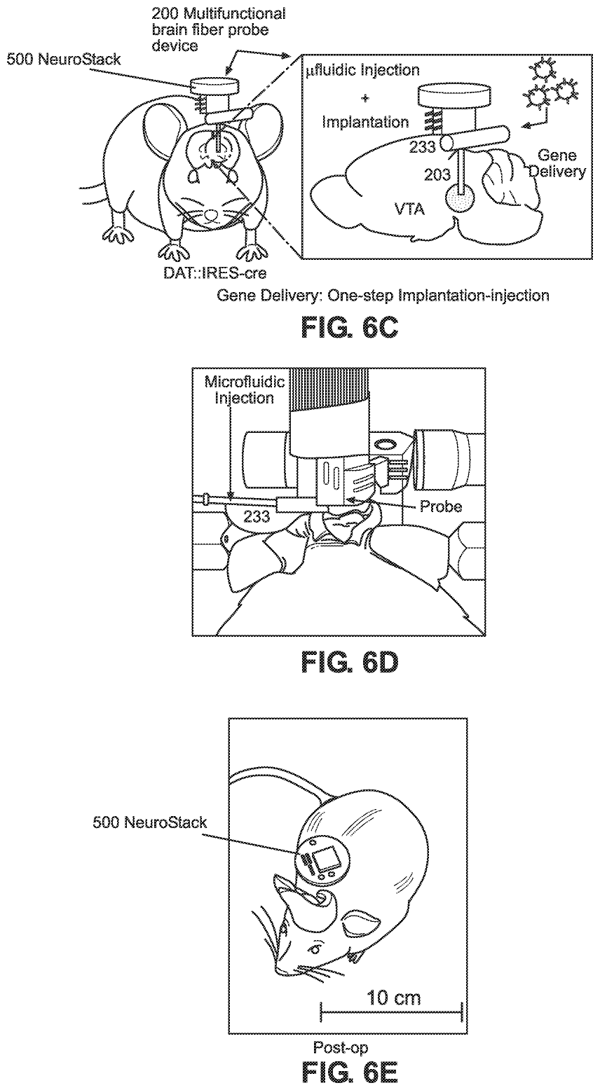

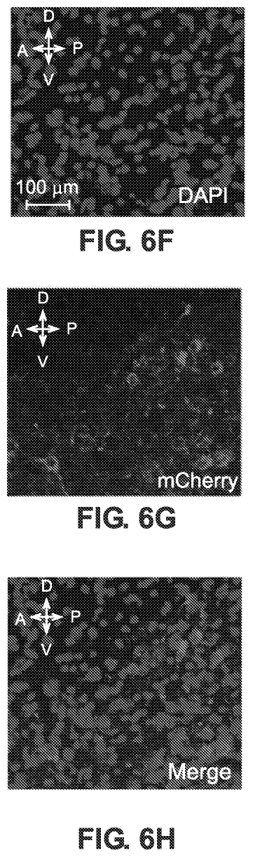

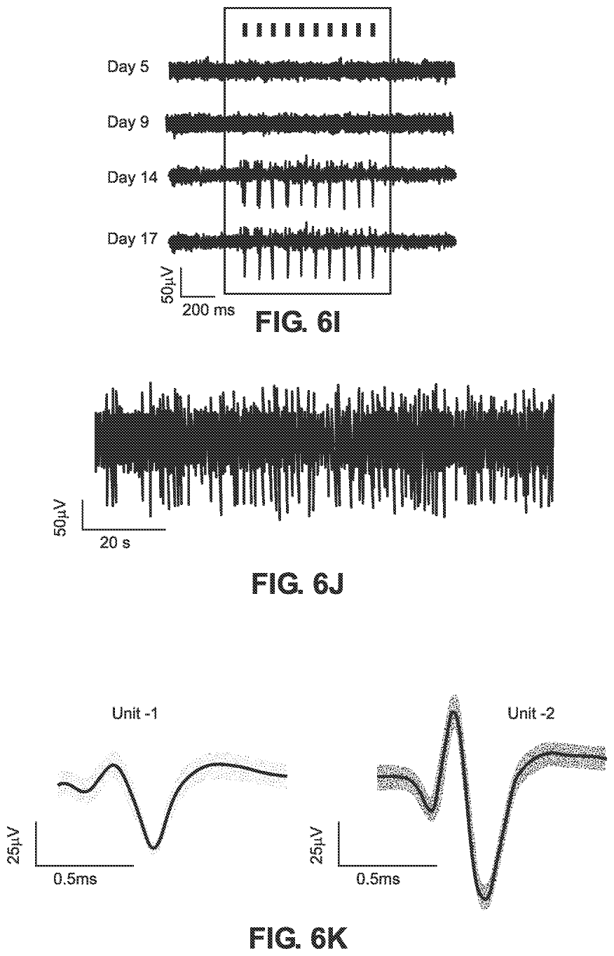

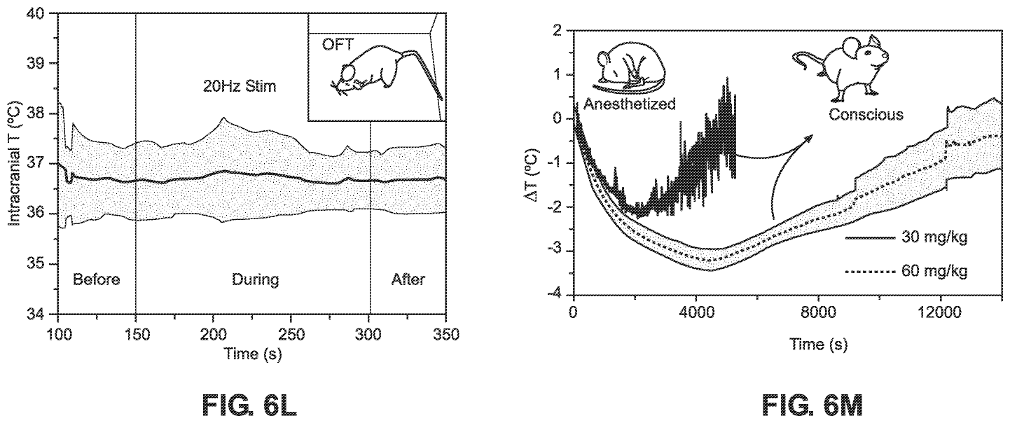

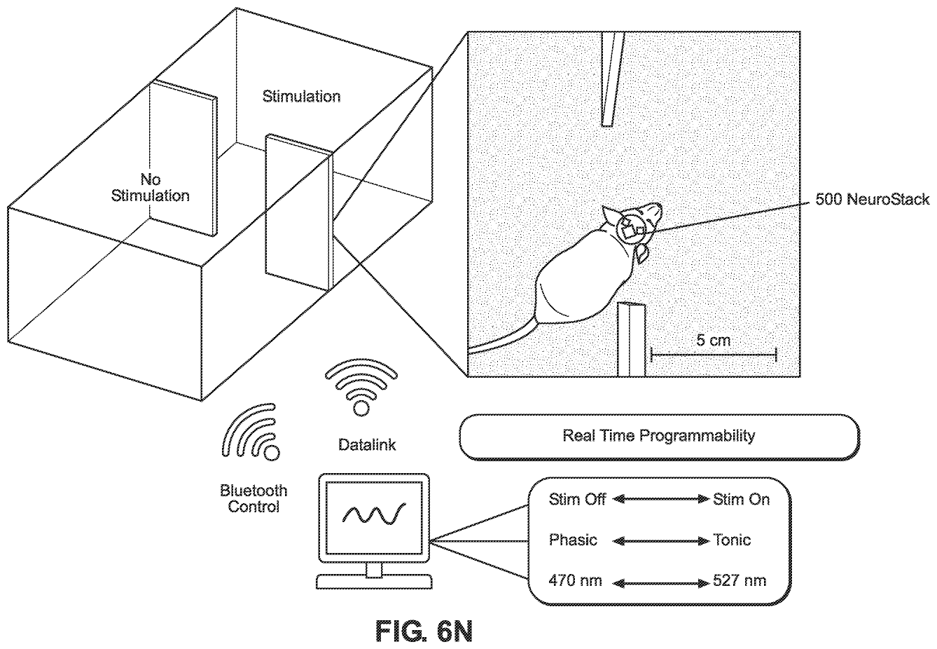

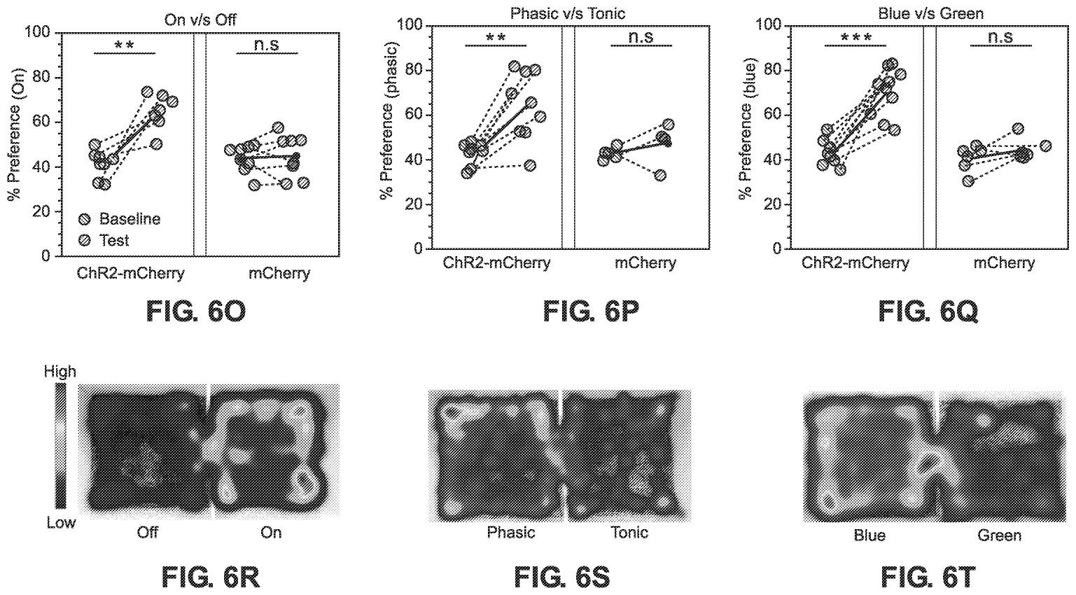

FIG. 6A shows a schedule for performing experiments that use the combined optical, electrical, fluidic, and thermal functionalities of a brain fiber probe 200 (FIGS. 2A-20) in a chronically implanted mouse. As an in-vivo validation testbed, we targeted the dopaminergic (DA) neurons in the ventral tegmental area (VTA) that are implicated in reward related behaviors. The brain fiber probe 200 was implanted in the VTA of Dopamine Transporter (DAT)::internal ribosomal entry site (IRES)-Cre transgenic mice (DAT::IRES-Cre) using standard stereotactic procedures. The integrated microfluidic channel in the fiber probe permitted delivery of a viral payload of AAV5-Efla-DIO-ChR2-mCherry to the VTA along with fiber probe implantation in a one-step process, shown in FIGS. 6B and 6C, thereby obviating multiple surgical interventions. FIGS. 6C-6E also show drawings of a mouse with a fully-assembled multifunctional brain fiber probe 200 with microfluidic tube 233, brain fiber 203, and NeuroStack module 500.

FIGS. 6D-6F show robust expression of ChR2 in coronal VTA sections. The μLED and tungsten microelectrodes in the same fiber probe allowed simultaneous electrical recording and optical stimulation of transfected neurons for validation of transgene expression. Since a number of neural recording electrodes exhibit optical stimulation artifacts, we first confirmed the physiological origin of our signals through in-vitro and in-vivo control experiments and devised artifact mitigation strategy by transient pulse shaping. The three modalities taken together uniquely enabled monitoring of the time course of Cre-dependent virus expression in DA cells as shown in FIG. 6I. Optically evoked, artifact-free multiunit neural activity was reliably recorded for up to two months in awake-behaving mice, as confirmed from the raw traces and PSTH profiles.

The thin, flexible fiber probes may show reduced relative micromotion in the brain tissue and can enable stable recording of single neuron activity over extended periods, which is relevant for many neurobiological studies. After confirming the functional stability of implanted electrodes over several weeks, we successfully recorded spontaneous neural activity from putative VTA neurons in chronically implanted mice (FIGS. 6J and 6K).

FIG. 6K shows recordings of brain electrical activity from the embedded tungsten electrode in the fiber probe. Two isolated units, Unit-1 and Unit-2, are two distinct neurons that the electrode could record from. This measurement is from one sensor (i.e., one recording electrode) on the same fiber probe. Their distinct waveforms (shapes) make them isolated units which allows us to say that most likely the electrical activity was from two distinct neurons firing in the vicinity of the electrode (distinct=isolated, units=neurons).

FIG. 6L shows intracranial temperature (36.6±0.6° C.) recorded with wireless thermal sensors. The temperature data do not change significantly during simultaneous wireless optical stimulation, thus confirming fiber probe device safety. The thermal sensors could also detect physiologically activated changes in intracranial temperature induced by an anesthetic drug mixture (Ketamine-Xylazine) that is known to trigger hypothermia by inhibiting thermoregulatory responses in a dose-dependent manner. In mice that were given intraperitoneal injection of Ketamine-Xylazine (9:1 dilution) at two concentrations (30 mg/kg and 60 mg/kg of Ketamine), the fiber probes recorded a robust drop in the temperature (FIG. 6M) at both dosages while the temperature recovery was time-locked to animal gaining consciousness and ambulating in the homecage. Multifunctional fiber probes with thermal sensors and recording electrodes can facilitate understanding of how changes in brain temperature alters neural dynamics under anesthesia.

FIG. 6N illustrates a real-time place preference (RTPP) task for mice transfected with ChR2 in the VTA that illustrates the wireless programmable photostimulation capability in the context of behavioral studies. The use of the BLE communication protocol with the NeuroStack module and fiber probe allows programmability of optical parameters without any line-of-sight or angular orientation handicap. To validate this, we performed RTPP tasks at three different stimulation conditions: (1) Stim On (25 Hz, 10 ms pulse, 1 s On duration, 2 s Off duration) v/s Stim Off; (2) Phasic stim (40 Hz, 5 ms pulse, 0.5 s On duration, 4 s Off duration) v/s Tonic stim (5 Hz, 10 ms pulse, ON); and (3) Blue stim (2=470 nm, 25 Hz, 10 ms, 1 s On duration, 2 s Off duration) v/s Green Stim (2=527 nm, 25 Hz, 10 ms, 1 s On duration, 2 s Off duration). FIG. 6N also shows NeuroStack 500 on the head of a mouse with implanted fiber probe. FIGS. 6O-6Q show that the mice with ChR2 expressed in VTA neurons exhibited a clear preference for the chamber paired with reinforcing stimulation condition as compared to their baseline values (pre-test day) which was absent in the control group of mCherry mice. FIGS. 6R-6T show the corresponding heat-maps tracing the position of the animal in a typical behavior run for each condition. We did not observe any significant differences in the average velocity or distance covered by fiber probe device-carrying mice compared to naïve un-operated counterparts. No significant changes in locomotor activity were observed in ChR2-mchery mice in response to VTA photostimulation. The fiber probe μLEDs also remained functional for up to 9-months post-implantation.

The biocompatibility of brain fiber probes was confirmed by immunohistochemical analysis of markers characteristic of glial scarring (activated macrophage marker ionized calcium-binding adaptor molecule 1 (Iba1), astrocytic marker glial fibrillary acidic protein (GFAP)). The immune response from polymer fiber probes was compared to that from a silica waveguide of comparable size (300 μm) at week-2 (P<0.05 for Iba1, P<0.001 for GFAP) and week-6 (P<0.05 for GFAP) post-implantation. We observed a consistently lower tissue response for polymer fiber probes at both the time points across all glial markers, which agrees with the reduced tissue micromotion predicted from FEM as well as lower bending stiffness observed experimentally.

### Multimodal Interrogation of Gut-Brain Communication in Awake-Behaving Mice

While techniques like optogenetics and pharmacology have revolutionized understanding of the brain, extending these methodologies to multimodal interrogation of gut neural circuits has remained out of reach. Our soft multifunctional GI fiber probes are capable of targeted light and chemical delivery along the GI tract and can address this challenge.

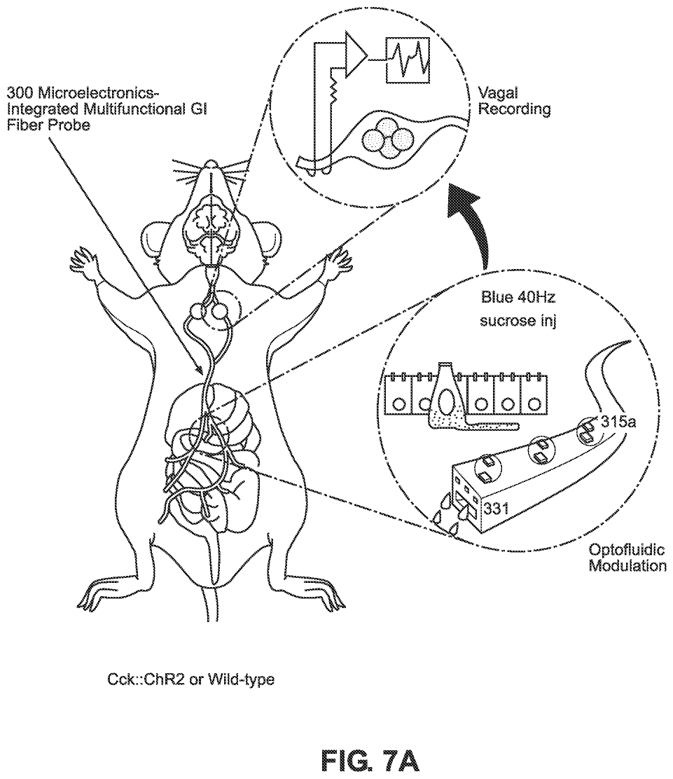

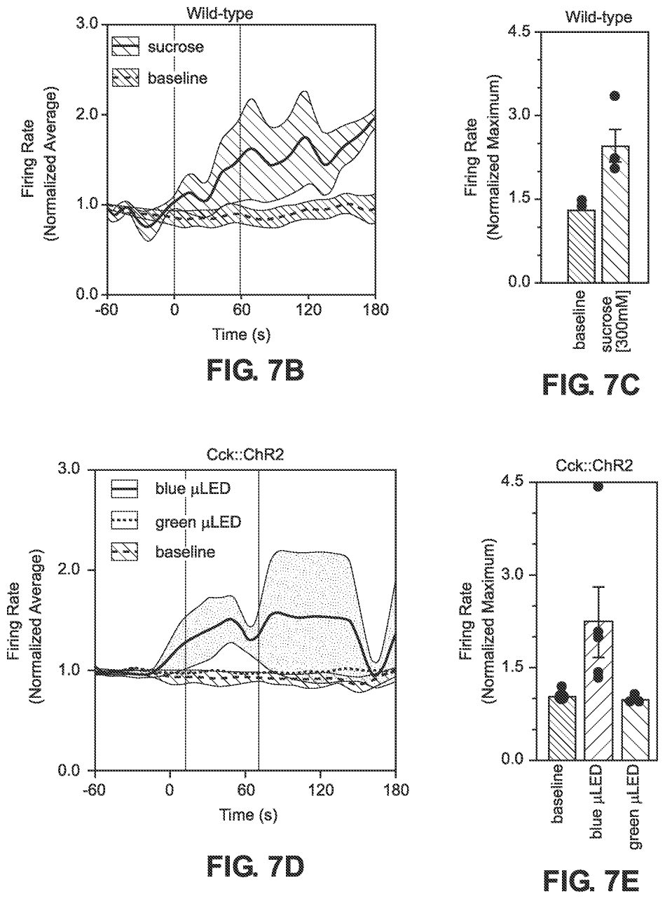

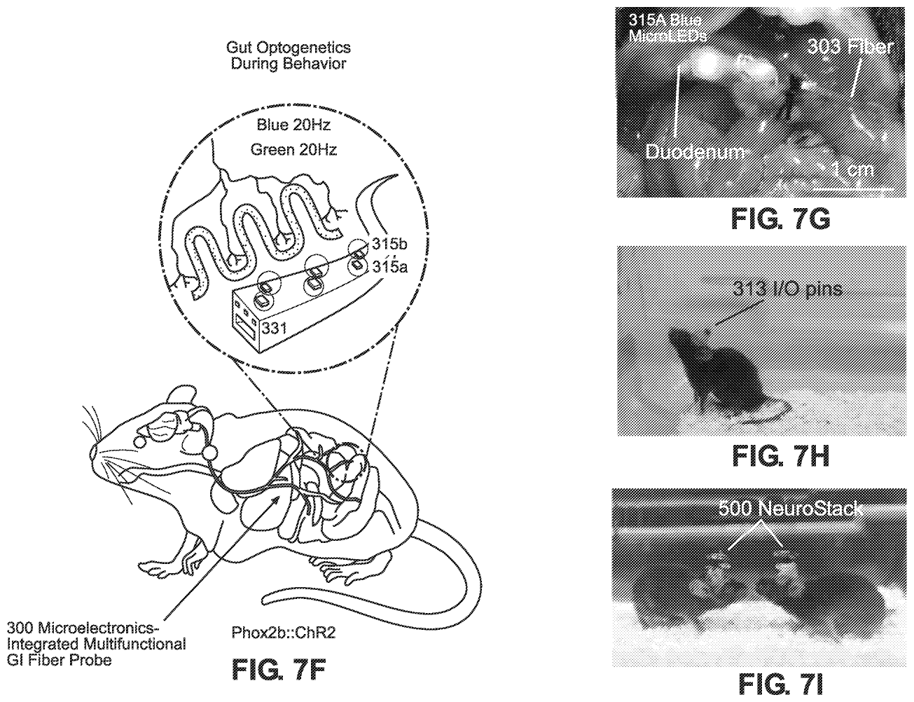

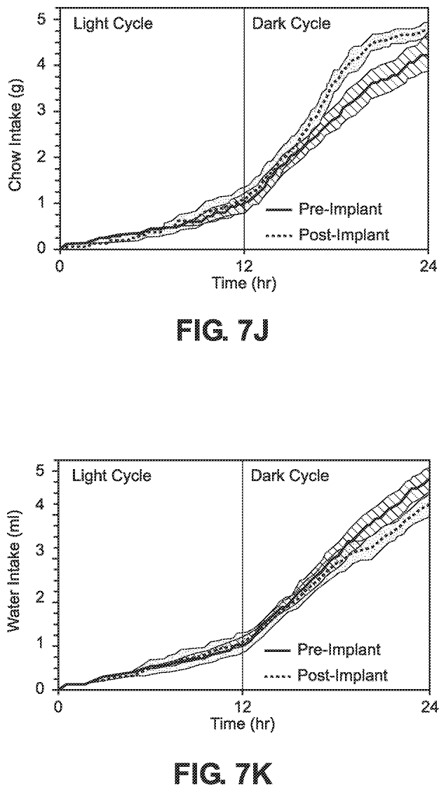

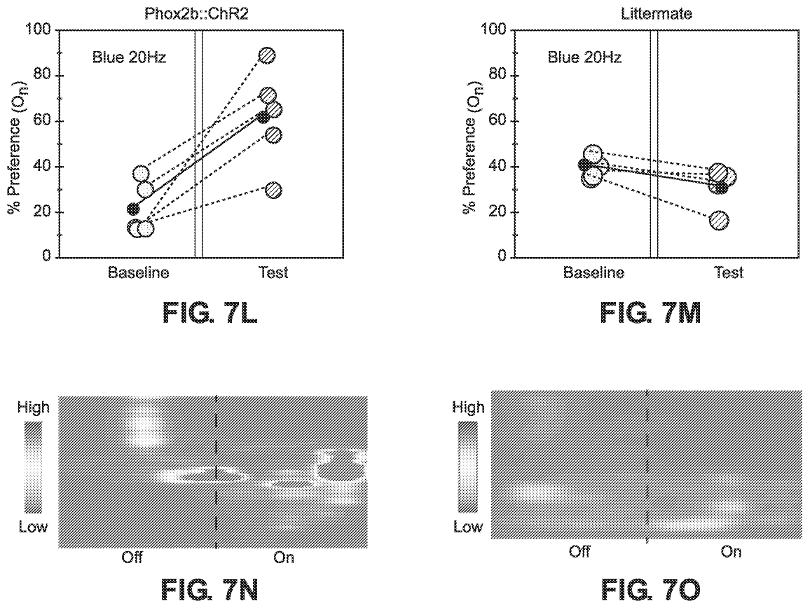

Neuropod cells are intestinal sensory epithelial cells that synapse with the vagal afferents and transduce signals to the brain-stem via the vagus nerve within milliseconds. We used this paradigm as a testbed to evaluate the dual opto-fluidic modality of fiber probes using cervical vagus nerve electrophysiology as a readout (FIG. 7A). The mouse drawings in FIGS. 7A and 7F also show the fully-assembled multifunctional GI fiber probe device 300, which has blue μLEDs 315a (FIGS. 7A and 7F), green μLEDs 315b (FIG. 7F), and microfluidic channel 331. In wild-type mice we found that intestinal infusion of sucrose solution (300 mM, 0.2 ml, 3.3 μl/s) via the microfluidic channels 331 of the implanted fiber probes 300 resulted in a significant increase in the vagal firing rate, as compared to the baseline activity (FIGS. 7B, C). This effect of chemical stimulation was recapitulated in transgenic mice expressing ChR2 in cholecystokinin (Cck) cells (Cck::ChR2), where optogenetic excitation via the blue μLEDs (40 Hz, 10 ms pulse width) within gut fiber probes resulted in an increased vagal firing rate. Vagal responses remained unchanged in Cck: ChR2 mice upon illumination with the green μLEDs integrated within the same fiber probes as well as in control littermates that lacked ChR2 in Cck cells (FIGS. 7D, E). Temperature can be stably measured from the lumen of the gut of wild-type mice during blue light stimulation trains.

Neuropod cells are expressed along the entire alimentary tract, where they release hormones and neurotransmitters to regulate food intake. To evaluate the impact of neuropod cells in feeding, we implanted the soft GI fiber probe in two distinct gastrointestinal regions: the duodenum and the ileum. The proper function of the intestinal lumen is critical for survival, so we first evaluated whether chronic implantation of the multifunctional fiber probes in the gut (FIG. 7G) and in the gut+brain affected animals' food and water intake and locomotor activity in the homecage. We found no significant changes in these characteristics before and after fiber probe implantation (FIGS. 7H-K), thereby confirming that the gut fiber probes do not obstruct the passage of ingested food or fluids or interfere with normal animal activity. FIGS. 7G-7I show the gut fiber probe 303 and blue μLEDs 315a implanted in the duodenum of a mouse, the I/O (input/output) pins 313 on NeuroStack 500.

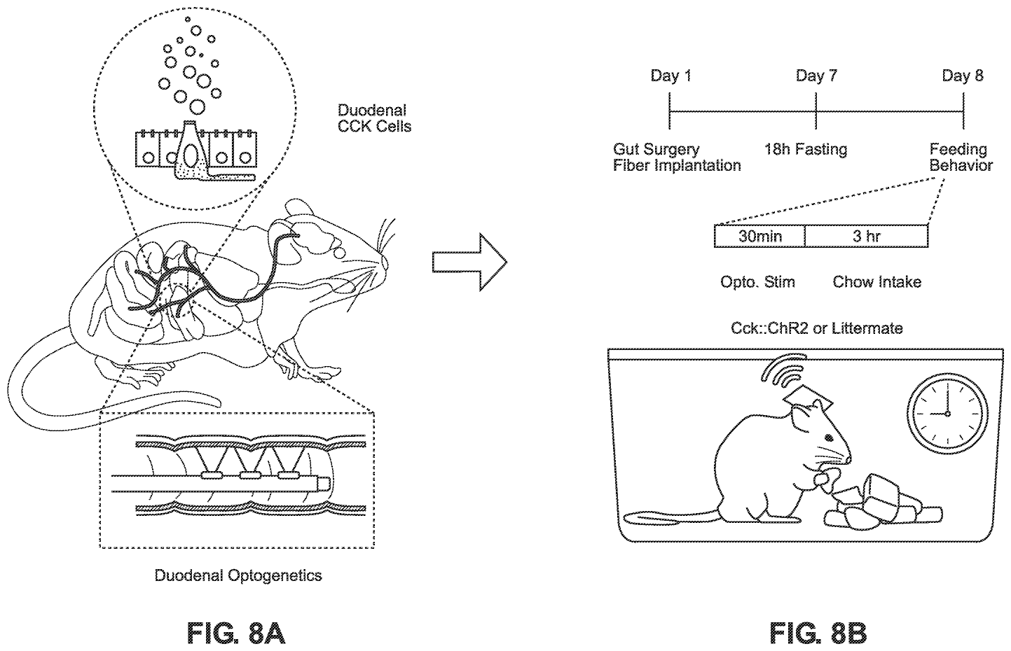

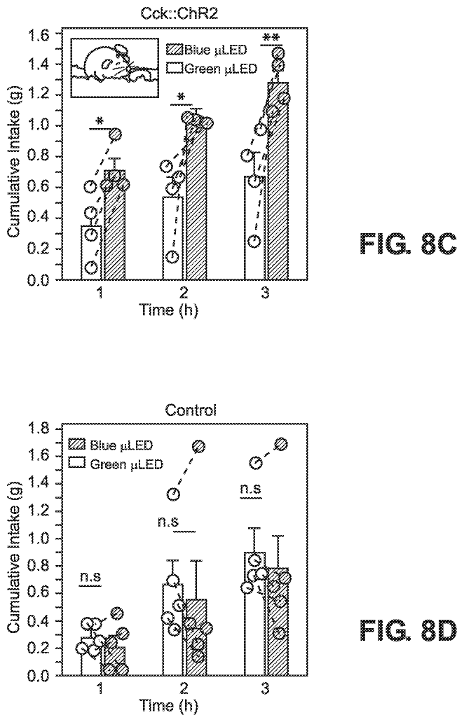

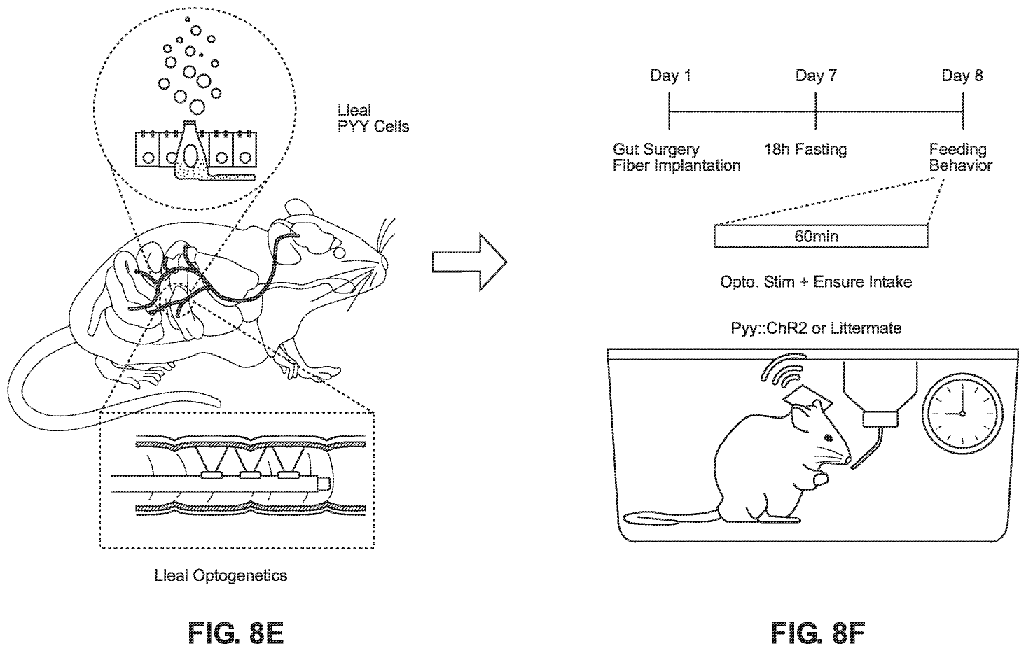

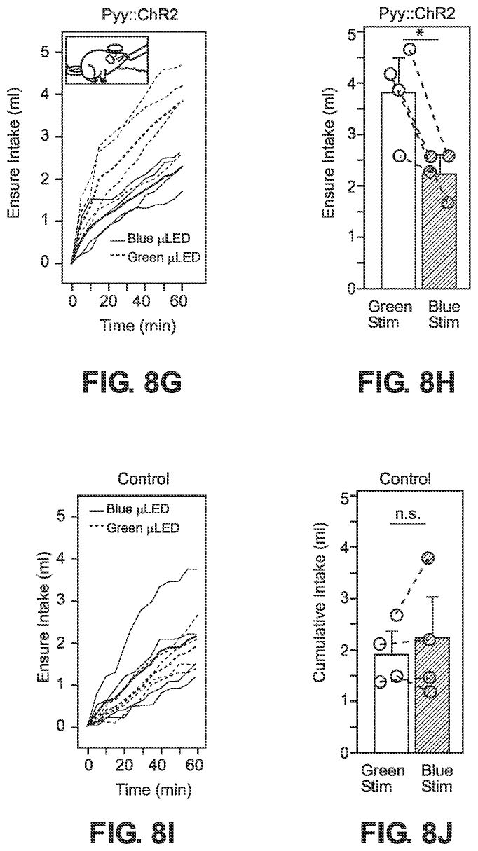

The soft GI fiber probe was implanted in the duodenum of Cck::ChR2 mice (n=4). A schematic illustration depicting optogenetic control of duodenal Cck+ cells with implanted gut fiber in the duodenum is shown in FIG. 8A, and the experimental timeline for evaluating feeding behavior in chronically implanted mice in a homecage feeding assay with ad-libitum access to standard chow is shown in FIG. 8B. In the duodenum, the release of the hormone cholecystokinin (CCK) suppresses food intake on the order of hours. After an overnight fast, stimulation of duodenal Cck cells with blue μLEDs (20 Hz, 10 ms pulse width, 0.5 s ON, Is OFF) significantly suppressed chow intake for three hours, compared to green light stimulation (FIG. 8C). Moreover, blue light stimulation had no impact on food intake in mice lacking ChR2 expression (FIG. 8D).

We then aimed to modulate food intake from a distinct GI region. In the ileum, the presence of fat and carbohydrates induces a process termed the ileal brake, caused by the release of the gut neuropeptides peptide YY (PYY) and glucagon-like peptide 1. The result is slowed gastric emptying, inhibited intestinal transit, and satiety. We reasoned that opotogenetic stimulation of neuropod cells in the ileum, genetically marked by Pyy, could stimulate the ileal brake, measured by a suppression of food intake. Thus, we implanted the soft GI fiber probe in the ileum of Pyy::ChR2 mice. A schematic illustration depicting optogenetic control of ileal Pyy+ cells with implanted gut fiber in the ileum is shown in FIG. 8E. The experimental timeline for evaluating feeding behavior in chronically implanted mice in a homecage feeding assay with ad-libitum access to Ensure solution (high fat and carbohydrate solution) is shown in FIG. 8F. Stimulation of ileal Pyy neuropod cells with blue μLEDs (20 Hz, 10 ms pulse width, 0.5 s ON, 1 s OFF) caused a significant suppression of the high fat and carbohydrate shake Ensure compared to green light (FIGS. 8G, 8H). Blue light stimulation in mice lacking ChR2 in Pyy ileal cells had no impact on intake (FIGS. 8I, 8J). Thus, neuropod cells can be leveraged along the gastrointestinal tract to regulate food intake.

Beyond satiety, the gut signals to the brain to induce motivation and reward. Our gut fiber probes provide an opportunity for testing whether functions of the central nervous system can be directly controlled from the intestine in awake-behaving mice (FIG. 7F). To test this hypothesis, we bred Phox2b::ChR2 mice using Cre-loxP recombination, in which ChR2 was expressed under the Phox2b promoter broadly found in vagal nodose neurons. Immunofluorescence imaging confirmed the presence of ChR2 in the vagal terminals that were tagged with the co-expressed tdTomato fluorescent marker.

Phox2b::ChR2 mice implanted with multifunctional gut fiber probes to the duodenum were subjected to an RTPP behavioral task during which intraluminal vagal stimulation with blue μLEDs (20 Hz, 10 ms pulse width, 0.5 s ON, Is OFF) caused a significant preference to the light-paired chamber compared to their pre-test values (FIG. 7L). The corresponding heat map depicting total time spent by a representative animal in both chambers appears in FIG. 7N. No significant differences in preference for either chamber was observed in control littermates lacking ChR2 expression that received identical optical stimulation (FIGS. 7M, O). Intraluminal vagal stimulation in Phox2b::ChR2 mice with green μLEDs (20 Hz, 10 ms pulse width, 0.5 s ON, Is OFF) also did not elicit any significant place preference to light paired chamber compared to their pre-test exploration. Thus, the soft, multifunctional, and wirelessly capable microelectronic fiber probes (microelectronics-integrated multifunctional fiber probes) are compatible with the anatomy and physiology of the gut lumen and can facilitate studies of the gut-brain pathways in the context of behavior.

### Methods

Multifunctional brain fiber probe (brain fiber probe) fabrication: The multifunctional brain fiber probes used for the experiments disclosed herein were produced by thermal drawing (in one implementation of the process, as shown in FIG. 2B) from a macroscopic model (multilayer preform layout for multifunctional brain fiber probe, as shown in FIG. 2A). The preform included three different layers and was produced through computer numerical control (CNC) milling of PC slabs 240a and 240b. The layer for convergence channels 211 was obtained by milling three square channels of 1.6 mm×1.6 mm with a pitch of 4 mm in a 14.8 mm×3 mm×30 cm PC slab. A PC top cover with dimensions of 14.8 mm×0.8 mm fully defined the convergence channels 211. The subsequent PC layer 240b had a large central channel (3.2 mm×2 mm×30 cm) that defined the microfluidic functionality (microfluidic channel 231) and was flanked by two additional smaller electrode channels 221 (1 mm×1 mm×30 cm) on either side that hosted ultra-fine tungsten microwires (25 μm) as recording electrodes for in-vivo electrophysiology. The tri-layered preform 201a was thermally consolidated at 185° C. for one hour in a vacuum oven (as shown in FIG. 2B) and subsequently drawn into ˜50 m long multifunctional fiber 203 at a size reduction ratio of ˜40 while simultaneously feeding spools of Ag—Cu and tungsten microwires, which serve as interconnects 212 and recording electrodes 222, respectively.

Multifunctional gut fiber probe (gut fiber probe) fabrication: Referring again to FIG. 3A, the preform assembly 301a for soft, multifunctional gut fibers 303 began with molding SEBS layers/pellets 350 into desired geometrical patterns in a CNC machined inverse aluminum mold 360. The top layer defined hollow channels 311 (3.6 mm×3.6 mm×30 cm) with a pitch size of 4 mm for hosting convergence microwires. The precursor to the microfluidic channel (2.8 mm×2 mm×30 cm) 331 and soft conducting electrodes with electrode channels 321 (2 mm×2 mm×30 cm) were incorporated in the bottom layer. The SEBS convergence channels 311 were lined with a U-shaped PC layer that had a wall thickness of 1 mm and channel size of 1.6 mm×1.6 mm. Finally, two slabs of a conducting polymer 322 (2 mm×2 mm×30 cm), namely carbon-loaded polyethylene (CPE), were inserted in the bottom layer that defined soft recording electrodes. This multilayered preform 301a was consolidated in a vacuum oven (130° C., 45 min) and subsequently drawn (in one implementation of the process, as shown in FIG. 3B) into meters-long multifunctional gut fibers 303 at a size reduction ratio of 40-45, while simultaneously feeding three spools of 40 μm Ag—Cu microwires 312.

### Microelectronics-Integrated Multifunctional Fiber Probe Fabrication

Multifunctional fiber probe fabrication: Fabrication of an implantable brain fiber probe 200 began with drawing a fiber preform 201a into a long (e.g., ˜50 m) fiber 203 as described above and for example as shown in FIG. 2B, then cutting the long fiber into 6 cm long segments. Next, the PC layer in the distal 1 cm of a fiber segment was dissolved away in dichloromethane for ˜2-3 min. This exposed the interconnect and electrode microwires in the fiber. The wires were subsequently soldered onto male header pins that were assembled inside a custom three-dimensional (3D) printed box (5 mm×7 mm×0.5 mm) and secured using UV curable epoxy. These header pins connect the implanted fiber probe to wired or wireless control modules in order to record and stimulate in freely behaving animals. About 0.5 cm of each interconnect wire was exposed by low-end machining with a razor blade at the distal end of the fiber under an optical microscope, followed by mounting of blue and green μLEDs chips 215a and 215b (as shown in FIGS. 2G-2I) using reflow soldering. The μLED chips were mounted along the length of the fiber probe 200 (before the distal 1 cm portion of the fiber probe 200). An insulated stainless-steel ground wire 214 connected to the header pin was soldered onto a ground screw.

Connection to microfluidic channel was established through a T-connection at the proximal end of the fiber probe 203 using soft access tubing 233. For this purpose, the microfluidic channel on the fiber probe was first exposed with a sharp razor blade cut and subsequently the fiber probe was threaded into the access tubing 233 through a metallic needle with an inner diameter similar to the fiber size. The T-junction was made water-tight by flowing UV-epoxy at the tubing-polymer junction. Successful microfluidic connection was confirmed by flowing a bolus of DI water. Finally, a 10 μm layer of vapor deposited parylene-C defined the bio-fluid barrier layer. The final fiber probe assembled in this way (shown in FIG. 2F) had an overall length of 4.5 cm.

A soft multifunctional gut/GI fiber probe can be made by drawing a preform 301a as described above (in one implementation of the thermal drawing process, as shown in FIG. 3B), segmenting the drawn fiber into shorter sections, then, for each section, exposing interconnect microwires, soldering to I/O pins, mounting of μLEDs, and connecting the microfluidic channel through a T-junction. The final multifunctional GI fiber probe (as shown in FIGS. 3E and 3F) had an overall length of 8.5 cm with three green μLEDs 315b and three blue μLEDs 315a hosted on the distal 2 cm of the fiber probe 300 at a separation of 1 cm (i.e., one blue μLED and one green μLED at the tip, one blue μLED and one green μLED about 1 cm from the tip, and one blue μLED and one green μLED about 2 cm from the tip). The gut fiber probe 300 was encapsulated in a ˜50-100 μm layer of medical grade silicone by inserting the fiber probe in a PTFE tubing, which acted as a sacrificial mold. The silicone mixture was filled and cured in the tubing and subsequently the tubing was cut open to yield a silicone-coated fiber probe. Finally, the microfluidic outlet was exposed 0.5 mm away from the foremost μLED.

Fiber probe characterization: For cross-sectional imaging of the fiber probes, samples from different sections of the draw (three each) were cold mounted into epoxy resin and subsequently polished on an automated grinding machine with series of progressively finer sandpapers. The polished epoxy blocks were imaged on a Carl Zeiss inverted microscope. The electrode impedance (n=3 fiber probes) was measured with a precision LCR meter (HP4284A, Agilent Technologies) with a sinusoidal input (10 mV, 20 Hz to 10 kHz). The optical characterization of fiber probe μLEDs (n=3 fiber probes) was performed by powering them with a DC power supply at different voltages and recording the light output with a photodetector (S121C, 400-1100 nm, 500 mW, Thorlabs) attached to a power meter (PM100D, Thorlabs). The I-V response of the μLEDs was measured with a potentiostat in a two-electrode configuration (Solartron).

We characterized the bending stiffness of the fiber probes with a dynamic mechanical analyzer (Q800, TA Instruments). Different samples (n=3 each) of 1.2 cm lengths were mounted in a single cantilever clamp and tested with a frequency sweep (0.1-10 Hz) under controlled displacement (20 μm) at 37° C. The cyclic bending tests (n=3 fiber probes) for soft gut fiber probes was performed with a mechanical testing machine (Z2.5 with testXpert III V1.11, Zwick/Roell) at varying displacements over 10⁴ cycles, while recording the light output from μLEDs at 2.7V after every decade. The diode-based thermal sensors (n=3 fiber probes) were calibrated against a commercial thermocouple by equilibrating the fiber probe and thermocouple on a hot plate at different temperatures and recording the current response using a potentiostat (Solartron) at 2.2 V forward bias.

The microfluidic capability of brain fiber probes (n=3) was evaluated by connecting the probes to an injection system NanoFil syringe, UMP-3, Word Precision Instruments) and flowing a DI water bolus at different injection speeds. The injection output was measured by weight and the injection rate was calculated by dividing the calculated injected volume by the time required to inject it. Identical procedure was employed for microfluidic characterization of gut fiber probes, except the injection of 0.2 ml bolus was performed manually over different times to yield required injection rates commensurate with intragastric infusions.

Optical simulations: Finite-elements simulation via COMSOL Multiphysics were used to calculate the optical field of the tissues. For absorbing-scattering media like biological tissues, the light transport equation was employed, where the light fluence rate at a given location in steady state Φ(r) obeys D∇²Φ(r)−μₐϕ(r)=0. D=(μₐ+μₛ′)/3 is the diffusion constant, μₐ is the absorption coefficient of the medium, and μₛ′ is the reduced scattering coefficient. Table 1 shows the absorption and reduced scattering coefficients of the brain and intestine at different excitation wavelengths.

TABLE 1
Absorption and reduced scattering coefficients

| | Excitation | Absorption | Reduced scattering |
|---|---|---|---|
| Medium | Wavelength (nm) | coefficient (cm⁻¹) | coefficient (cm⁻¹) |
| Brain | 475 | 3.67 | 51.15 |
| Brain | 532 | 3.84 | 46.3 |
| Intestine | 475 | 26.3 | 33.25 |

The brain was modeled as a homogeneous medium with absorption and reduced scattering coefficient specified in Table 1. The dimension of the brain model was set to be large such that the radiative power decays to zero before reaching to the boundary of the brain. To model the power of μLED, an omnidirectional plane source is inserted at the center of the brain. The power of the source is defined to be two times the actual power to account for the emission directivity of the μLED. The intestine is modeled as two coaxial cylinders filled with chyme, with the outer one being the serosoal membrane (r=1.8 mm) and the inner one being the mucosal membrane (r=1.5 mm). The μLED is placed at the center of the coaxial structure. As the chyme does not scatter light, the region of the gut wall is employed for the light transport equation. The μLED emission profile is fitted by a power cosine function. Light emitted from the center of μLED is propagated to the inner surface of the intestine to determine the light fluence rate at the inner walls, which is used as a boundary source to calculate the power distribution in the intestine region.

Thermal simulations: Finite-elements simulation via COMSOL Multiphysics was used to determine the temperature profile of tissues upon excitation of the LEDs. The transient heat transport equation in a biological tissue can be expressed as

$$ρ {C}_{p} \frac{\partial T}{\partial t} + ρ {C}_{p} u \cdot ∇ T - k {∇}^{2} T = Q + {Q}_{bio} ,$$

where ρ, k, Cₚ, and u are the density, thermal conductivity, heat capacity, and fluid velocity, respectively. Q_{bio} represents a volumetric bioheat source term that can be further expressed as Q_{bio}=ρ_{b}C_{p,b}ω_{b}(T_{b}−T₂)+Qₘₑₜ. T_{b},C_{p,b},ωₚ,ρ_{b},Qₘₑₜ are the arterial blood temperature, specific heat of blood, blood perfusion rate, blood density, and metabolic heat source, respectively. Table 2 lists the parameters used for evaluating Q_{bio}.

TABLE 2
Parameters for metabolic heat source.

| | Arterial | Specific | | | Metabolic |
|---|---|---|---|---|---|
| | blood | heat, | Blood | Blood | heat |
| | temper- | blood | perfusion | density | source |
| Medium | ature (K) | (J/(kg K)) | rate (s⁻¹) | (kg/m³) | (W/m³) |
| Brain | 310.15 | 36000 | 0.008 | 1057 | 9132 |
| Intestine | 310.15 | 36000 | 0 | 1057 | 9132 |

In addition to the metabolic heat source, there are two other sources of heat that originate from the μLED. The first heat source is due to the absorbance of optical power in the tissues, and the second heat source is directly from the inefficiency of the μLED. For modeling heat transport in the brain, the first term is introduced through coupling the heat transfer module with the radiation in absorbing-scattering medium module in COMSOL, and the second term is introduced by defining a surface heat source at the μLED surfaces. The temperature profile in the gut was obtained without considering the optical absorption term due to the low optical power at the serosoal membrane and high light transmission through the chyme region. In addition, heat generated by blood perfusion was not considered in the intestine tissue.

Mechanical simulation: Finite element modeling software (Abaqus) was used for the simulation of the bending of the different fibers and the fiber displacement in moving tissue. The element used for the materials in the fiber, such as the cladding polymer, conducting polymer, and metal microwires, was C3D8H with a HEX element shape. The element used for the tissue was C3D10H with a TET element shape. For the bending fiber simulation, the fiber probe was held fixed at one end and was displaced with a fixed distance at the other end. The amount of displacement varied with different curvature radius. For the simulation of implants in the brain tissue, the tissue at its bottom was displaced sideward with a distance of 0.1 mm, while the exposed end of the fiber probe was fixed.

NeuroStack wireless module: A NeuroStack module is composed of a custom printed circuit board (PCB) with an NRF52 microcontroller for BLE communication with the central system (nRF52840 DK development kit) that is connected to a base station computer. A male header pin near the edge of the circular board allows the device to be connected to and disconnected from the implanted multifunctional fiber probe. Two vertical header pins on the base of the board allow for the attachment and removal of the optional modules, including the intensity module. For this study, optional modules for precise intensity control were used across different trials.

To prepare the individual devices and optional modules, components were hand mounted onto the custom PCBs using reflow soldering and software was loaded using J-Link programmers with an Arduino library. The fiber probe μLEDs were driven by either a constant 3.3 V source or a programmable DAC, with a current-limiting series resistor in place to keep the brightness within the desired level. For wireless recording, the transmitted data was collected using a 12-bit ADC and sent over BLE to a central collection point. To improve the bandwidth and reduce packet overhead, the data was batched at the cost of latency in the recordings. The data received was transferred over serial to the MATLAB program or saved for later analysis.

Validation of the device waveforms was done using an oscilloscope to compare measured frequencies and shapes to those specified in the interface. The current consumption patterns were first characterized using a Keithley 100B source meter. A further test of battery life was conducted by running the device with the desired stimulation parameters until the output voltage was below the μLED turn-on voltage. For testing the temperature recording module, the fiber probe was placed on a hotplate with a commercial thermocouple and was left to settle for 30 seconds between each temperature reading. Recordings of the amplifier output voltage and the temperature from the commercial sensor were compared to allow for calibration.

Surgical implantation of multifunctional fiber probes in the brain (brain fiber probes): All animal procedures were approved by the MIT Committee on Animal Care and carried out in accordance with the National Institutes of Health Guide for the Care and Use of Laboratory Animals. C57BL/6 mice aged 6-8 weeks (Jackson Laboratory) and transgenic DAT::Ires-Cre mice aged 6-10 weeks were used for the study and all surgeries were conducted under aseptic conditions. Mice were anaesthetized using via intraperitoneal (IP) injection of ketamine/xylazine mixture in saline (in mg/kg bodyweight: ketamine, 100; xylazine, 10), and then positioned in a stereotactic frame (David Kopf Instruments). A skin incision was made to expose the skull. Lambda and bregma points were used to align the skull with respect to the Mouse Brain Atlas. All implantation and injection coordinates were established according to the brain atlas.

A single step injection/implantation was performed in the VTA (VTA, coordinates relative to bregma; −3.2 mm anteroposterior (AP); 0.5 mm mediolateral (ML); −4.4 mm dorsoventral (DV)). Adeno-associated viruses serotype 5 (AAV5) carrying EfIα-DIO-hChR2-mCherry and Efla-DIO-mCherry plasmids were purchased from University of North Carolina Vector Core at concentrations of 2× 10¹² particles/mL and 3×10¹² particles/ml, respectively. Using a standard microinjection apparatus (NanoFil Syringe and UMP-3 Syringe pump, Word Precision Instruments) 0.8 μl of virus was injected through the microfluidic channels of implanted fiber probes at an infusion rate of 150-300 nl/min. During injections, the fiber probes were raised 0.1 mm to accommodate the virus volume. The stainless-steel ground screw was affixed to the skull on the contralateral hemisphere. Finally, the fiber probe was fixed to the skull with layer of adhesive (C&B Metabond; Parkell) and dental cement (Jet-Set 4, Lang Dental). Following the surgery and recovery, mice were single housed and were maintained at 22° C. and a 12-hour light/dark cycle and provided with food and water ad libitum.

Surgical implantation of multifunctional fiber probes in the gut (gut fiber probes): All animal procedures were approved by the Duke University Institutional Animal Care and Use Committee and carried out in accordance with the National Institutes of Health Guide for the Care and Use of Laboratory Animals. Adult wild-type, Phox2b::ChR2, Pyy::ChR2, or Cck::ChR2 mice or littermates were anesthetized with isoflurane (1-3% in oxygen). A 1 cm incision was made from the xiphoid process diagonally to left-mid clavicular line. The peritoneal cavity was accessed, and the stomach extra-corporealized for implantation in wild-type, Phox2b::ChR2 and Cck::ChR2 mice. In these mice, the distal end of the fiber probe containing the μLEDs was introduced to the duodenum through the pylorus. To access the pylorus, a purse string suture was made in the gastric antrum, between which a small incision was made in the stomach wall. The distal end of the fiber probe was threaded into the proximal duodenum. The purse string stitch was then tied to secure the fiber probe in the intestine. In Pyy::ChR2 mice, a 1 cm incision was made 3 cm below the xiphoid process to access the cecum. The cecum was extra-corporealized and the distal end of the fiber probe embedded with the μLEDs was introduced into the distal ileum through a purse string suture, as in the duodenal surgeries. The purse string suture was tied to secure the fiber probe in the ileum. In all surgeries, the remaining length of the fiber probe was tunneled to the base of the skull. The peritoneum and overlying skin were sutured closed. The fiber probe exited the tunnel at the base of the skull. The skull was etched with a scalpel blade and a thin layer of Metabond cement (Clear L-powder S399+catalyst; Metabond) was applied. Then, the Metabond layer was etched and the fiber probe was attached using standard dental cement (Stoelting #51458). Mice recovered for at least 5 days during which they were fed wet mash and received appropriate post-operative care. Inclusion criteria at the end of each study were that the fiber probe was appropriately secured in the proximal small intestine and that the μLEDs were operational.

Surgical implantation of dual multifunctional fiber probes in the brain and in the gut (dual brain-gut multifunctional fiber probes, or helical fibers): Wild-type mice (n=6) were implanted with the dual brain-gut multifunctional fiber probes (multisite gut-brain fiber probes). The gut μLED fiber probe was implanted into the duodenum, as described above. Subsequently, the mouse was placed into a stereotaxic frame and the brain fiber probe was directed to the VTA, as described above.

In-vivo electrophysiology: Multifunctional fiber probes were attached to PZ2-32 head stage connected to RZ5D electrophysiology system (Tucker Davis Technologies, TDT). Optogenetic stimulation pulses were delivered with integrated blue or green μLEDs. For all experiments, pulsed stimulation with 5 ms pulse width was used. Stimulation was delivered in 1-s stimulation epochs separated by 4-second rest epochs. Following recording, electrophysiological signal was digitized with 50 kHz sampling frequency and filtered in the frequency range 0.3-5 kHz. Subsequent signals processing and analysis was done with Matlab (Mathworks). Spiking activity was detected using threshold detection with a threshold set at five standard deviations from the mean of the signals, with a downtime of 2 ms to reject double detections. Clustering and classification of spikes were performed by principal component analysis (PCA) and Gaussian mixture model (GMM) clustering (with full and independent covariance matrices). The quality of the clustered data was assessed by calculating the L-ratio and the isolation distance of the classified clusters.

In-vivo impedance spectroscopy: To assess the stability of tungsten microelectrodes in-vivo, we performed electrochemical impedance spectroscopy of tungsten electrode v/s ground screw in VTA implanted BL/6 mice for up to 6 months (n=3 mice) with a portable BioLogic VMP3 potentiostat.

In-vivo intracranial temperature measurements: Wireless intracranial temperature measurements were performed in an open field test. BL/6 mice (n=6) implanted in the VTA with multifunctional fiber probes (pre-calibrated) were coupled to NeuroStack module and allowed to explore an open field arena (30 cm×30 cm) as they received wireless photostimulation (20 Hz, 10 ms pulse width) with implanted blue μLED for 200 seconds. Real-time feed on intracranial temperature was received throughout the session. Anesthesia induced brain hypothermia was quantified by i.p. injection of a mixture of Ketamine-Xylazine (9:1 dilution) at 30 mg/kg and 60 mg/kg dosage in n=3 VTA implanted BL/6 mice. The temperature measurements were performed until the animal gained consciousness and began freely moving in the homecage.

In-vivo gut temperature measurements: Wireless gut temperature measurements were performed in a clean homecage with food and water removed. A wild-type mouse was implanted with a μLED embedded gut fiber probe to the duodenum. Temperature was continually recorded for 10 minutes, with blue light pulsed.

In-vivo gut optofluidic modulation with simultaneous vagal cuff electrophysiology: Whole nerve recordings were performed in Cck::ChR2 mice. A soft multifunctional GI fiber probe with two connected tubes for PBS perfusion and stimulant delivery was surgically inserted through the stomach wall into the duodenum. A perfusion exit incision was made at the ligament of Treitz for the small intestine. To control for volume pressure and to act as a within subject baseline, PBS was constantly perfused through the isolated intestinal region at about 400 μL per minute. Stimulation conditions were applied after recording 2 minutes of baseline activity. During nutrient stimulation conditions, PBS perfusion was continuous and 200 μL of stimulant was perfused over 1 minute using a syringe pump (Fusion 200, Chemyx). The 1-minute infusions of each ligand were separated by at least 6 minutes, or the return to baseline firing rate, whichever came first. Sucrose [300 mM] was used as the nutrient, as it stimulates the vagal firing rate. Blue light (λ=470 nm, 20 Hz, 30.3 mW/mm², 10 ms pulse width) or green light (2=527 nm, 20 Hz, 45.6 mW/mm², 10 ms pulse width) was delivered concomitant with the sucrose infusion. Extracellular voltage was recorded. The raw data were analyzed using SpikeTailor, a custom MATLAB software (MathWorks) script. Spikes were detected using a threshold detected based on RMS noise. The firing rate was calculated using a Gaussian kernel smoothing algorithm in 200 ms bins.

Real time place preference assay in brain and gut implanted mice: Behavioral tests were performed by an investigator with knowledge of the identity of the experimental groups versus control groups. Brain. DAT::IRES-Cre mice injected in the VTA in a one-step implantation/injection procedure (AAV5-EF1a-DIO-hChR2-mCherry or AAV5-DIO-mCherry; n=8-10/group) were handled and acclimated to the investigator for 5 mins each prior to the beginning of the behavior. The mice were then acclimated to NeuroStack by connecting the module to the I/O pins of the implanted fiber probe and then allowed to explore their homecage for 15 minutes. On the following day (pre-test day), NeuroStack module carrying mice were allowed to freely explore an unbiased two-compartment chamber (60 cm×30 cm×30 cm) for 30 minutes while being video recorded. The time spent by each animal in both the chambers was calculated with commercial software (Ethovision XT Noldus). Mice that showed >70% preference to a chamber in the baseline explorations were discarded from subsequent analysis.

On the day of the test, the less preferred chamber for each animal was coupled to wireless photostimulation condition by controlling NeuroStack from a base computer. The live video feed from a recording camera provided input on the animal location and the investigator controlled the status of the stimulation condition in real-time. Three different photostimulation conditions were tested using the above procedure: (1) blue stim ON (25 Hz, 10 ms pulse width, Is ON, 2 s OFF) v/s blue stim OFF; (2) Phasic stim (40 Hz, 5 ms pulse width, 0.5 s ON, 4 s OFF) v/s tonic stim (5 Hz, 1 ms pulse width, ON); and (3) blue stim (25 Hz, 10 ms pulse width, Is ON, 2 s OFF) v/s green stim (25 Hz, 10 ms pulse width, Is ON, 2 s OFF).

For gut testing, Phox2b::ChR2 mice and their negative genotype littermates were implanted with the soft GI fiber probe to the duodenum. Mice were acclimated to investigator handling and connection to the NeuroStack module as in the brain experiments. On the day before the test, NeuroStack module was attached the the I/O pins of the implanted fiber probe and mice were allowed to freely explore an unbiased chamber (Techniplast Greenline IVC cage for mice) for 20 minutes. Animal activity was determined by beams crossed in the x and y planes and was collected with a 100 Hz scan rate using the TSE PhenoMaster software. On test day, the less preferred chamber for each mouse was coupled to wireless photostimulation by controlling the wireless module from a base computer. The live activity feed provided input on mouse location in real-time and the investigator controlled the stimulation in real-time. There were three different conditions: (1) The experimental group was Phox2b::ChR2 mice with blue stim ON (20 Hz, 10 ms pulse width) vs OFF; (2) The LED control group was Phox2b::ChR2 mice with green stim ON (20 Hz, 10 ms pulse width) vs OFF; and (3) the genetic control group was negative genotype littermates with blue stim ON (20 Hz, 10 ms pulse width) vs OFF.

Chow intake in mice duodenal implanted with gut fiber probe: Cck::ChR2 mice or negative controls were implanted with the soft GI fiber probes in the duodenum. Mice were acclimated to the investigator handling and connection to the NeuroStack module. Mice were food deprived overnight (18 h) before connection to the NeuroStack module and 30-minutes of light pulses (20 Hz, 10 ms pulse width). After 30-minutes of stimulation, mice were disconnected from NeuroStack and given access to standard show pellets (Purina 5001). Chow intake was measured each hour for 3 hours. Mice had ad libitum access to water for the duration of the food restriction and testing. Each mouse received blue-light and green-light stimulation, randomized per condition. At least 48-h separated each test day.

Ensure intake in mice ileal implanted gut fiber probe: Pyy::ChR2 mice or negative controls were implanted with the soft GI fiber probes in the ileum. Mice were acclimated to experimenter handling and connection to the NeuroStack module. Mice were acclimated to Ensure (30%) solution for 6 hours following surgical recovery and at least 48 hours before the first experimental session. Mice were food deprived overnight (18 h) before connection to the NeuroStack module. Mice received one-hour of light pulses (20 Hz, 10 ms pulse width), which began 10 minutes before access to the Ensure solution. Ensure (30%) was loaded into 5 mL serological pipettes were fashioned as sippers. Mice had access to the solution for 1-h and intake was measured every 5 minutes. Mice did not have access to food or water during the test sessions. Each mouse received blue-light and green-light simulation, randomized per condition. At least 48 hours separated each test day.

Locomotor tests on brain and gut implanted mice: To test whether brain, gut or gut-brain dual implants coupled to NeuroStack impacted locomotion, we evaluated locomotor behavior over 20 minutes. Open field test on naïve (un-operated) and implanted mice carrying NeuroStack were conducted in an open chamber (60 cm×30 cm×30 cm) for brain implantations and in the homecage for gut and gut-brain dual implantations over 20 min. Locomotor activity was recorded as described in the real time place preference assay. The mouse position, distance traveled, and speed, were calculated from the locomotor and/or activity measurements.

Food intake and water intake of gut and gut-brain implanted mice: Animals were housed individually in a custom-built PhenoMaster behavioral phenotyping system (TSE Systems Inc. Chesterfield, MO). The PhenoMaster was programmed (software version 7.1.1) to automatically maintain a light cycle (0300 lights on; 1500 lights off), temperature control (22° C.), and humidity control (50%). Animals were provided with standard mouse chow (Purina 5001) and reverse osmosis water ad libitum. Food hopper and water bottle were attached to weight sensors (TSE) that made automatic measurements every 5 seconds to the nearest 0.01 g. For drinking measurements, a 10-second smoothing interval with a maximum raw analog-to-digital conversion counts difference of 40,000 was permitted. For weight measurements, a 15-second smoothing interval with a 15 g threshold and a maximum raw analog-to-digital conversion counts difference of 1,000,000 was permitted. Intake was measured every 5 seconds. Data were corrected for minor fluctuations by permitting only a monotonically increasing function for both food and water intake: values that represented negative food intake were replaced by the most recent value. For stability of intake measurements, the mean intake and water intake was calculated for two consecutive days for each individual mouse.

Immunohistochemical evaluation of foreign body response in brain: Animals (n=5 BL/6 mice bilaterally implanted in VTA) were anesthetized with isoflurane, injected with fatal plus (100 mg kg-1 IP), and transcardially perfused with 50 mL of ice-cold PBS followed by 50 mL of ice-cold 4% paraformaldehyde (PFA) in PBS. The fiber probe devices were carefully explanted and the brains were removed and fixed in 4% PFA in PBS for 24 h at 4° C., then stored in PBS afterward. Coronal slices (50 μm thickness) were prepared using a vibratome (Leica VT1000S) and a razor blade (Electron Microscopy Sciences, 72002) in ice-cold PBS. The slices were then stored in PBS at 4° C. in the dark until staining. Slices were permeabilized with 0.3% v/v Triton X-100 and blocked with 2.5% donkey serum in PBS for 30 min. Slices were incubated overnight at 4° C. in a solution of 2.5% donkey serum in PBS and a primary antibody (Iba1: Goat anti-Iba1, ab107159 Abcam, 1:500 dilution; GFAP: Goat anti-GFAP, ab53554 Abcam, 1:1000 dilution). Following incubation, slices were washed three times with PBS. The slices were then incubated with secondary antibody (Donkey anti-Goat Alexa Fluor 488, A11055, 1:1000, Thermofischer) for two hours at room temperature on a shaker followed by three more washes with PBS. Slices were then incubated with DAPI (4′6-diamidino-2-phenylindole) (1:50 000) for another 20 minutes and washed three times with PBS. Fluoromount-G (SouthernBiotech) was used for mounting slices onto glass microscope slides. A laser scanning confocal microscope (Fluoview FV1000, Olympus) was used for imaging with 20× objectives, with z-stack images across the slice thickness. A region of interest for imaging the immune response was chosen based on the fiber probe implantation location.

Immunohistochemical evaluation of foreign body response in the small intestine: Phox2b::tdTomato mice were transcardially perfused with PBS for 3 minutes followed by 4% PFA for 3 minutes at a rate of 600 μl/min. Each small intestine was harvested, opened lengthwise, rolled with the proximal end in the center, and post-fixed in 4% PFA for 3 hours. Tissue was then dehydrated in 10% sucrose for 1 hour and 30% sucrose for at least 12 hours. Samples were embedded in OCT (VWR) and stored at −80° C. Tissue was sectioned onto slides at 16 μm using a cryostat. Tissue slides were post-fixed in 10% normal buffered formalin (VWR) for 10 minutes then washed in tris-buffered saline with 0.05% Tween-20 (TBST) (Sigma). Tissue was then washed with TBST, stained with DAPI (1:4000) for 3 minutes, washed in TBST, and mounted using Fluoro-Gel with Tris Buffer (Electron Microscopy Sciences). Imaging was done on a Zeiss 880 Airyscan inverted confocal microscope. Images were adjusted for brightness/contrast using ImageJ (Fiji).

Immunohistochemical evaluation of foreign body response: Wild type mice (n=5 per group) bilaterally implanted in the VTA with brain fiber probes or commercial silica waveguides (300 μm, FT300UMT Thorlabs) were anesthetized with isoflurane, injected intraperitoneally with fatal plus (100 mg kg⁻¹), and transcardially perfused with 50 mL of ice-cold PBS followed by 50 mL of ice-cold 4% paraformaldehyde (PFA) in PBS. The fiber probes/waveguides were carefully explanted and the brains were removed and additionally fixed in 4% PFA in PBS for 24 h at 4° C., then stored in PBS afterward. Coronal slices (50 μm thickness) were prepared using a vibratome (Leica VT1000S) and a razor blade (Electron Microscopy Sciences, 72002) in ice-cold PBS. The slices were then stored in PBS at 4° C. in the dark until staining. Slices were permeabilized with 0.3% v/v Triton X-100 and blocked with 2.5% donkey serum in PBS for 30 min. Slices were incubated overnight at 4° C. in a solution of 2.5% donkey serum in PBS and a primary antibody (Iba1: Goat anti-Iba1, ab107159 Abcam, 1:500 dilution; GFAP: Goat anti-GFAP, ab53554 Abcam, 1:1000 dilution). Following incubation, slices were washed three times with PBS. The slices were then incubated with a secondary antibody (Donkey anti-Goat Alexa Fluor 488, A11055, 1:1000, Thermofischer) for 2 h in room temperature on a shaker followed by additional three washes with PBS. Slices were then incubated with DAPI (4′ 6-diamidino-2-phenylindole) (1:50 000) for another 20 minutes and washed three times with PBS. Fluoromount-G (SouthernBiotech) was used for mounting slices onto glass microscope slides. A laser scanning confocal microscope (Fluoview FV1000, Olympus) was used for imaging with 20× objectives, with z-stack images across the slice thickness. Region of interest was chosen based on the implant location, imaging the immune response in the device surrounding.

Hematoxylin and eosin stain evaluation of foreign body response: Duodenal tissue from wild-type mice naïve (n=4) and implanted (n=4) to the duodenal implant was sliced and evaluated for hematoxylin and cosin (H&E) by the Duke Pathology Histology Lab. The villus height and crypt depths were measured using ImageJ 2 (1.5.3). For each mouse, the villus height and crypt depth were calculated as the average of 10 villi or crypts.

Statistical analysis: OriginPro or JMP Pro 15 software was used to assess the statistical significance of the comparison studies in this work. Power analyses for determining sample sizes of immunohistochemistry and behavior tests were not performed. Instead, the group sizes were chosen based on previous research conducted in the same brain circuit or intestinal region. This enabled direct comparison of our results with the prior work. In the statistical analysis of fiber probe characterization, one-way ANOVA followed by Tukey's post-hoc comparison test was used with thresholds of *P<0.05, **P<0.01, ***P<0.001. For the comparison between two groups in immunohistochemistry analyses, behavior assays and hypothermia effect quantification paired t-test were used, and significance threshold was placed at *P<0.05, **P<0.01, ***P<0.001. For parametric tests, data distribution was assumed to be normal. Shaded areas and error bars in the figures represent standard deviation.

### CONCLUSION

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain, using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A multifunctional fiber probe comprising:
a polymer fiber segment having (i) a proximal end and (ii) a distal end configured to be inserted into tissue;
a microfluidic channel running along a length of the polymer fiber segment and having a first opening at the proximal end of the polymer fiber segment and a second opening at the distal end of the polymer fiber segment and configured to convey fluid between the first opening and the second opening;
electrodes running along the length of the polymer fiber segment and configured to make electrophysiology measurements of the tissue;
electrical interconnects running along the length of the polymer fiber segment;
solid-state devices disposed along the length of the polymer fiber segment or disposed along a distal portion of the polymer fiber segment in electrical communication with the electrical interconnects and configured to interact with the tissue; and
a control module at a proximal end of the polymer fiber segment and connected to the electrical interconnects and the electrodes and configured to record the electrophysiology measurements and to control the solid-state devices.

2. The multifunctional fiber probe of claim 1, wherein the polymer fiber segment comprises at least one of polycarbonate (PC) or styrene-ethylene-butylene-styrene (SEBS).

3. The multifunctional fiber probe of claim 1, wherein the polymer fiber segment has a Young's modulus of about 2.3 GPa to about 3.0 GPa.

4. The multifunctional fiber probe of claim 1, wherein the polymer fiber segment has a Young's modulus of about 5 MPa to about 30 MPa.

5. The multifunctional fiber probe of claim 1, wherein the solid-state devices comprise light-emitting diodes configured to optogenetically modulate tissue with pulses of green light and/or blue light.

6. The multifunctional fiber probe of claim 1, wherein the solid-state devices comprise temperature sensors configured to measure a temperature of the tissue around the distal portion of the polymer fiber segment.

7. The multifunctional fiber probe of claim 1, wherein the control module comprises a wireless transceiver configured to receive commands from an external device and/or to transmit data to the external device.

8. The multifunctional fiber probe of claim 1, further comprising:
a stretchable interconnect connecting the proximal end of the polymer fiber segment to the control module.

9. The multifunctional fiber probe of claim 8, wherein the stretchable interconnect comprises a helically coiled fiber with embedded microwires in electrical communication with the electrical interconnects and the control module.

10. The multifunctional fiber probe of claim 8, wherein the polymer fiber segment and at least a part of stretchable interconnect are configured to be implanted in the tissue.

11. The multifunctional fiber probe of claim 8, wherein the polymer fiber segment is a first polymer fiber segment, the electrical interconnects are first electrical interconnects, and the tissue is brain tissue, and further comprising:
a second polymer fiber segment configured to be inserted into gastrointestinal tissue;
second electrical interconnects running along a length of the second polymer fiber segment, and
wherein the control module is operably coupled to the second electrical interconnects.

12-20. (canceled)
